(19) United States
(12) Patent Application Publication
(10) Pub. No.: US 2023/0366036 A1
(43) Pub. Date: Nov. 16, 2023

(54) METHODS OF DETECTING RESIDUAL DISEASE AND TREATMENT THEREOF

(71) Applicants: Aadel Chaudhuri, St. Louis, MO (US); Pradeep Chauhan, St. Louis, MO (US)

(72) Inventors: Aadel Chaudhuri, St. Louis, MO (US); Pradeep Chauhan, St. Louis, MO (US)

(73) Assignee: Washington University, St. Louis, MO (US)

(21) Appl. No.: 18/316,294

(22) Filed: May 12, 2023

Related U.S. Application Data
(60) Provisional application No. 63/341,543, filed on May 13, 2022.
(60) Provisional application No. 63/403,230, filed on Sep. 1, 2022.

(51) Int. Cl. C12Q 1/6886; G16B 20/20; G06N 20/00; G16B 30/10; G16B 40/20

(52) U.S. Cl. CPC C12Q 1/6886; G16B 20/20; G06N 20/00; G16B 30/10; G16B 40/20; C12Q 2600/118; C12Q 2600/156

(57) ABSTRACT

Low-pass whole genome sequencing, as well as targeted hybrid-capture next-generation sequencing, were performed to detect both small mutations and genome-wide copy number alterations to more precisely detect MRD after physician's-choice neoadjuvant chemotherapy. A machine learning model using both cell-free DNA and pre-treatment clinical characteristics is disclosed. With this method, it was possible to significantly predict both progression-free and overall survival.

### CROSS-REFERENCE TO RELATED APPLICATIONS

This application claims priority from U.S. Provisional Application Serial Nos. 63/341,543 filed on 13 May 2022 and 63/403,230 filed on 1 Sep. 2022, which are incorporated herein by reference in their entireties.

### STATEMENT REGARDING FEDERALLY SPONSORED RESEARCH OR DEVELOPMENT

Not applicable.

### MATERIAL INCORPORATED-BY-REFERENCE

Not applicable.

### FIELD

The present disclosure generally relates to methods of detecting residual disease resulting from urinary tract-associated cancer using cell-free DNA analysis.

### BACKGROUND

Molecular residual disease (MRD) detection during and after treatment is very challenging to detect using current methods in clinical arsenals. Without accurate assessment, patients are either over- or under-treated. In bladder cancer, the radical cystectomy surgery is extensive, morbid, life-altering, and can potentially be deadly.

### SUMMARY

In an aspect of the present disclosure, a method of detecting residual disease in a subject having or suspected of having a urinary tract-associated cancer is provided. The method comprises: obtaining a urine sample from the subject; extracting cell-free DNA (cfDNA) from the urine sample; detecting a cfDNA-derived metric using ultra-low-pass whole genome sequencing (ULP-WGS) and next-generation sequencing (NGS); wherein the cfDNA-derived metric comprises at least one of a tumor fraction (TFx) value, a variant allele frequency (VAF) value and a tumor mutational burden (TMB) value; and determining the subject to have residual disease or no residual disease based on the cfDNA-derived metric.

In some embodiments, the NGS comprises urine cancer personalized profiling by deep sequencing (uCAPP-Seq), the determining comprises employing a machine learning model based on the cfDNA-derived metric, detecting the cfDNA-derived metric further comprises detecting single nucleotide variants (SNVs) or copy number alterations (CNAs) in the cfDNA, and/or the cfDNA-derived metric consists of a TFx value, VAF value, and a TMB value. In some embodiments, the machine learning model comprises a random forest model and is further based on a clinical variable selected from the group consisting of age, gender, ethnicity, smoking status, receipt of chemotherapy, tumor invasion status, and combinations thereof. In some embodiments, the urinary tract-associated cancer is a bladder cancer, for example, a muscle-invasive bladder cancer. In some embodiments, a cancer treatment was administered to the subject prior to obtaining the urine sample, and wherein the cancer treatment is a chemotherapy, a radiotherapy, or an immunotherapy. In some embodiments, determining the subject to have residual disease comprises determining a negative predictive value (NPV) of at least about 70%, a positive predictive value (PPV) of at least about 60%, or an area under the curve (AUC) of at least about 0.70. In some embodiments, the machine learning model further predicts overall survival (OS) or progression-free survival of the subject based on the cfDNA-derived metric.

In another aspect of the present disclosure, a method of treating a subject having or suspected of having a urinary tract-associated cancer is provided. The method comprises: obtaining a urine sample from the subject; extracting cell-free DNA (cfDNA) from the urine sample; detecting a cfDNA-derived metric using ultra-low-pass whole genome sequencing (ULP-WGS) and next-generation sequencing (NGS); wherein the cfDNA-derived metric comprises at least one of a tumor fraction (TFx) value, a variant allele frequency (VAF) value and a tumor mutational burden (TMB) value; and determining the subject to have residual disease or no residual disease based on the cfDNA-derived metric; and providing: a cancer treatment to the subject if the subject is determined to have residual disease, or active surveillance to the subject if the subject is determined to have no residual disease.

In some embodiments, the NGS comprises urine cancer personalized profiling by deep sequencing (uCAPP-Seq), the determining comprises employing a machine learning model based on the cfDNA-derived metric, detecting the cfDNA-derived metric further comprises detecting single nucleotide variants (SNVs) or copy number alterations (CNAs) in the cfDNA, and/or the cfDNA-derived metric consists of a VAF value, a TFx value, and a TMB value. In some embodiments, the machine learning model is further based on a clinical variable selected from the group consisting of age, gender, ethnicity, smoking status, receipt of chemotherapy, tumor invasion status, and combinations thereof. In some embodiments, the urinary tract-associated cancer is a bladder cancer, and wherein the cancer treatment comprises a chemotherapy, a radiotherapy, an immunotherapy, or a surgical treatment, for example, a cystectomy. In some embodiments, a cancer treatment was administered to the subject prior to obtaining the urine sample.

Other objects and features will be in part apparent and in part pointed out hereinafter.

### DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1 is an exemplary embodiment showing pathologic complete response prediction using a random forest model based on urine tumor DNA in accordance with the present disclosure. Urine was collected prospectively from 74 localized bladder cancer patients pre-operatively on the day of curative-intent radical cystectomy after physician's choice neoadjuvant treatment. Urine cell-free DNA was sequenced by uCAPP-Seq (for single nucleotide variants) and ULP-WGS (for genome-wide copy number alterations) and then correlated with residual tumor in the surgical resection specimen and with patient survival.

FIG. 2 is a study schema in accordance with the present disclosure. Patients with localized bladder cancer who were candidates for radical cystectomy were prospectively enrolled in this study. Urine samples were then collected for uCAPP-Seq and ULP-WGS analysis as shown in the schema. Urine samples from healthy adults were also used for ULP-WGS and uCAPP-Seq analysis.

FIG. 3A-FIG. 3C is an exemplary embodiment showing genome-wide copy number plots with annotation of genes important in bladder cancer in accordance with the present disclosure. Plots represent the aggregate copy number alterations compiled from urine cell-free DNA data in (FIG. 3A) patients with no pCR (n=39), (FIG. 3B) patients with pCR (n=35) or (FIG. 3C) healthy adults (n=15). Each panel depicts log₂ copy number ratios across the genome. Red represents copy number gain while blue represents copy number loss. Annotated genes are those previously reported in TOGA to be copy-number altered in bladder cancer.

FIG. 4A-FIG. 4B is an exemplary embodiment showing subject characteristics and detected genomic alterations in accordance with the present disclosure. FIG. 4A and FIG. 4B contain co-mutation plots showing genomic alterations (mutations and copy number alterations) detected in pre-operative urine cell-free DNA from each patient with no pCR versus pCR (FIG. 4A) and healthy adults (FIG. 4B). Mutational data represent non-silent SNVs detected within the MRD uCAPP-Seq gene panel, while copy number alterations represent ultra-low-pass whole genome sequencing data, focusing on genes reported by TOGA to be altered in muscle-invasive bladder cancer. Patient and healthy donor characteristics are represented by the upper heatmaps.

FIG. 5 is an exemplary embodiment showing pathologic complete response prediction using a random forest model based on urine tumor DNA in accordance with the present disclosure. FIG. 5 contains a scatter plot showing SNV-derived maximum VAFs.

FIG. 6A-FIG. 6B is an exemplary embodiment showing performance of LAPP-Seq in matched urine and plasma samples for detecting MRD and predicting pathologic response in accordance with the present disclosure. FIG. 6A is a scatter plot of maximum VAF levels after square-root transformation in urine versus plasma from 40 localized bladder cancer patients, compared to gold-standard surgical pathology. FIG. 6B shows ROC analysis for classifying pCR from no pCR patients by CAPP-Seq. CAPP-Seq in urine cell-free DNA classified pathologic response more accurately than in paired plasma (AUC 0.78 versus 0.62).

FIG. 7A-FIG. 7B is an exemplary embodiment showing pathologic complete response prediction using a random forest model based on urine tumor DNA in accordance with the present disclosure. FIG. 7A and FIG. 7B contain scatter plots showing inferred tumor mutational burden (FIG. 7A) and CNA-derived tumor fraction levels (FIG. 7B) in urine cell-free DNA from patients with localized bladder cancer. Scatter plots display these three different urine cell-free DNA metrics, stratified by pathologic complete response status, with significance determined by the Mann-Whitney U-test. VAF and CNA-derived tumor fraction data are shown after square root transformation.

FIG. 8 is an exemplary embodiment showing a random forest model with LOOCV to predict pathologic complete response status in accordance with the present disclosure. FIG. 8 contains a schema depicting the model's development, validation, and application.

FIG. 9A-FIG. 9B is an exemplary embodiment showing pathologic complete response prediction using a random forest model based on urine tumor DNA in accordance with the present disclosure. FIG. 9A shows a ROC analysis of random forest model integrating urine tumor DNA metrics and other pretreatment clinical variables (see e.g., FIG. 8 and FIG. 10). ROC curve demonstrating the model's performance for predicting pCR after LOOCV (AUC=0.80, p<0.0001). FIG. 9B is a stacked bar plot depicting NPV and PPV of the random forest model with LOOCV, with significance determined by the Fisher's exact test.

FIG. 10 is an exemplary embodiment showing a random forest model with LOOCV to predict pathologic complete response status in accordance with the present disclosure. FIG. 10 is a bar graph showing importance of features in the random forest model used for predicting pCR status. Error bars represent the standard deviation.

FIG. 11A-FIG. 11B is an exemplary embodiment showing a random forest model based on urine cell-free DNA features with LOOCV to predict pathologic complete response status in accordance with the present disclosure. FIG. 11A is a bar graph showing importance of features in the random forest model used for predicting pCR status based on urine cell-free DNA features only (TFx, maximum VAF and iTMB). Error bars represent the standard deviation. FIG. 11B shows ROC analysis of random forest model for predicting pCR after LOOCV (AUC=0.76, p=0.0001).

FIG. 12A-FIG. 12D is an exemplary embodiment showing survival analysis comparing urine MRD detection to pathologic analysis of the resection specimen in accordance with the present disclosure. FIG. 12A and FIG. 12B contain Kaplan-Meier plots showing progression-free survival (FIG. 12A) and overall survival (FIG. 12B) stratified by MRD detection in urine, determined by the LOOCV random forest model (see e.g., FIG. 8 and FIG. 10). FIG. 12C and FIG. 12D contain Kaplan-Meier plots showing progression-free survival (FIG. 12C) and (FIG. 12D) overall survival stratified by pCR determined by microscopic analysis of the radical cystectomy specimen. Survival times shown are relative to the time of radical cystectomy. p values were calculated by the log-rank test and HRs by the Mantel-Haenszel method.

FIG. 13A-FIG. 13G is an exemplary embodiment showing LOOCV random forest model applied to MIBC patients to predict pathologic response and survival outcomes in accordance with the present disclosure. FIG. 13A-FIG. 13C contain scatter plots displaying (FIG. 13A) maximum VAF (square-root transformed), (FIG. 13B) iTMB, and (FIG. 13C) TFx (square-root transformed), stratified by pathologic response status among MIBC patients (n=58), with significance determined by the Mann-Whitney U test. FIG. 13D shows ROC analysis demonstrating the LOOCV random forest model's performance in classifying MIBC patients by pCR status; AUC of 0.80 (p=0.0001). FIG. 13E is a stacked bar plot depicting NPV and PPV of the LOOCV random forest model with significance determined by the Fischer's exact test. FIG. 13F and FIG. 13G contain Kaplan-Meier curves showing (FIG. 13F) progression-free survival and (FIG. 13G) overall survival based on the LOOCV random forest model applied to patients with MIBC (n=58). p values were calculated by the log-rank test and HRs by the Mantel-Haenszel method.

FIG. 14A-FIG. 14E is an exemplary embodiment showing LOOCV random forest model applied to MIBC patients who received neoadjuvant chemotherapy to predict pathologic response and survival outcomes in accordance with the present disclosure. FIG. 14A-FIG. 14C contain scatter plots displaying (FIG. 14A) maximum VAF (square-root transformed), (FIG. 14B) iTMB, and (FIG. 14C) TFx (square-root transformed), stratified by pathologic response status among MIBC patients who received NAC (n=38). Significance was determined by the Mann-Whitney U test. FIG. 14D and FIG. 14E contain Kaplan-Meier curves of (FIG. 14D) progression-free survival and (FIG. 14E) overall survival based on the LOOCV random forest model applied to MIBC patients who received NAC. P values were calculated by the log-rank test and HRs by the Mantel-Haenszel method.

FIG. 15A-FIG. 15B is an exemplary embodiment showing random forest model evaluated for survival outcomes in a held-out validation cohort in accordance with the present disclosure. FIG. 15A and FIG. 15B contain Kaplan-Meier curves showing (FIG. 15A) progression-free survival and (FIG. 15B) overall survival in a held-out validation cohort of 45 localized bladder cancer patients, after random forest model training using data from 29 localized bladder cancer patients. p values were calculated by the log-rank test and HRs by the Mantel-Haenszel method

### DETAILED DESCRIPTION

The present disclosure is based, at least in part, on the discovery that molecular residual disease (MRD) can be detected in bladder cancer patients using low-pass whole genome sequencing (ULP-WGS), hybrid-capture next generation sequencing (e.g., uCAPP-Seq), and a machine learning algorithm. Methods are described herein that obtain urine samples from subjects, extract cell-free DNA, detect a cfDNA-derived metric comprising a variant allele frequency (VAF) value, a tumor fraction (TFx) value, or a tumor mutational burden (TMB) value, and employ a machine learning model to determine whether a subject has or does not have residual disease based on the cfDNA-derived metric. As described herein, determining whether a subject has or does not have residual disease may be used to decide an appropriate course of treatment for the subject, e.g., whether active surveillance is appropriate or whether the subject would benefit from additional treatment, such as surgery.

For example, the disclosed methods more precisely determine which subjects would benefit from radical cystectomy versus who can be safely watched after neoadjuvant chemotherapy. The method also identifies high-risk patients who would benefit from additional treatment, even after surgery, e.g., with adjuvant immunotherapy.

Disclosed herein is the performance of low-pass whole genome sequencing (ULP-WGS) as well as targeted hybrid-capture next-generation sequencing (NGS, for example uCAPP-Seq) to detect both small mutations and genome-wide copy number alterations to more precisely detect MRD after physician's-choice neoadjuvant chemotherapy. A machine learning model was then developed using both cell-free DNA and pre-treatment clinical characteristics. With this method, it was possible to significantly predict both progression-free and overall survival.

As described herein, VAF and TF values are measured. VAF or TF greater than a healthy subject or control, can be predictive of unfavorable outcomes (e.g., PCR, reduced PFS, reduced OS, or MRD). For example, VAF or TF can have values of about 0.0%; about 0.02%; about 0.03%; about 0.04%; about 0.05%; about 0.06%; about 0.07%; about 0.08%; about 0.09%; about 0.1%; about 0.2%; about 0.3%; about 0.4%; about 0.5%; about 0.6%; about 0.7%; about 0.8%; about 0.9%; about 1%; about 2%; about 3%; about 4%; about 5%; about 6%; about 7%; about 8%; about 9%; about 10%; about 11%; about 12%; about 13%; about 14%; about 15%; about 16%; about 17%; about 18%; about 19%; about 20%; about 21%; about 22%; about 23%; about 24%; about 25%; about 26%; about 27%; about 28%; about 29%; about 30%; about 31%; about 32%; about 33%; about 34%; about 35%; about 36%; about 37%; about 38%; about 39%; about 40%; about 41%; about 42%; about 43%; about 44%; about 45%; about 46%; about 47%; about 48%; about 49%; about 50%; about 51%; about 52%; about 53%; about 54%; about 55%; about 56%; about 57%; about 58%; about 59%; about 60%; about 61%; about 62%; about 63%; about 64%; about 65%; about 66%; about 67%; about 68%; about 69%; about 70%; about 71%; about 72%; about 73%; about 74%; about 75%; about 76%; about 77%; about 78%; about 79%; about 80%; about 81%; about 82%; about 83%; about 84%; about 85%; about 86%; about 87%; about 88%; about 89%; about 90%; about 91%; about 92%; about 93%; about 94%; about 95%; about 96%; about 97%; about 98%; about 99%; or about 100%. Recitation of each of these discrete values is understood to include ranges between each value. Recitation of each range is understood to include discrete values within the range.

Urinary Tract-Associated Cancer

The methods described herein may be used for treating a subject having or suspected of having cancer, or detecting residual disease resulting from a cancer. For example, the cancer can be a cancer associated with the urinary tract.

The methods described here can be useful for any urothelial carcinoma (also known as transitional cell carcinoma (TCC)) at any part of the urinary tract. For example, the urothelial carcinoma can be all types of bladder cancers, genitourinary cancers, cancers of the urinary tract, kidney cancers, ureteral cancers, or renal pelvis cancers. Examples of kidney cancers can be renal cell carcinoma (e.g., kidney cancer types: sarcomatoid (e.g., clear cell, papillary, chromophobe), medullary, collecting duct, urothelial carcinoma, sarcoma, Wilms tumor, lymphoma, oncocytoma, angiomyolipoma).

Genitourinary cancer can be bladder cancer, kidney cancer, testicular cancer, urethral cancer, prostate cancer, penile cancer, urethral cancer, or adrenal cancer. Genitourinary cancer types can include adrenal cancer (e.g., adrenocortical carcinoma), bladder cancer (e.g., urothelial carcinoma, urachal cancer), kidney cancer (e.g., collecting duct carcinoma, renal cell carcinoma, rhabdoid tumor of the kidney, Renal pelvic cancer (ureteral cancer), Wilms tumor (nephroblastoma)), penile cancer (e.g., urethral cancer), prostate cancer, testicular cancer (germ cell tumor) (e.g., seminoma, non-seminoma, such as choriocarcinoma proud or teratoma).

As another example, the cancer associated with the urinary tract can be bladder cancer, kidney cancer, ureter cancer, urothelial carcinoma, squamous cell carcinoma, adenocarcinoma papillary carcinomas, flat carcinomas, localized bladder cancer, metastatic bladder cancer, muscle-invasive bladder cancer (MIBC), non-muscle-invasive bladder cancer (NMIBC), or urothelial carcinoma with trophoblastic differentiation, including choriocarcinomatous differentiation.

Cancer Treatment

Described herein are methods of treating a subject having or suspected of having a urinary tract-associated cancer. The methods herein may be used to determine the appropriate cancer treatment for a subject, such as a surgical treatment, radiotherapy, chemotherapy, and whether a neoadjuvant and/or adjuvant therapy should be administered. The methods herein may also be used to determine whether a subject is eligible for active surveillance in lieu of a cancer treatment.

Neoadjuvant generally refers to a treatment administered prior to surgery, with the goal of shrinking a tumor or stopping the spread of cancer to make surgery less invasive and more effective. Adjuvant therapy can be administered after surgery to kill any remaining cancer cells with the goal of reducing the chances of recurrence. The methods herein may be used to detect residual disease after, for example, administering a neoadjuvant therapy to a subject, and thus may also be used to determine whether a subject should receive adjuvant therapy.

Chemotherapy

The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Standard of care for bladder cancers currently can include nivolumab, pembrolizumab, atezolizumab, enfortumab-vedotin, erdafitinib, methotrexate, vinblastine, doxorubicin, cisplatin, and gemcitabine (and combinations thereof). Examples of other chemotherapeutic agents can include alkylating agents such as thiotepa and cyclophosphamide; alkyl sulfonates such as busulfan, improsulfan and piposulfan; aziridines such as benzodopa, carboquone, meturedopa, and uredopa; ethylenimines and methylamelamines such as altretamine, triethylenemelamine, trietylenephosphoramide, triethiylenethiophosphoramide and trimethylolomelamine; acetogenins (such as bullatacin and bullatacinone); a camptothecin (such as the synthetic analogue topotecan); bryostatin; callystatin; CC-1065 (such as its adozelesin, carzelesin and bizelesin synthetic analogues); cryptophycins (particularly cryptophycin 1 and cryptophycin 8); dolastatin; duocarmycin (such as the synthetic analogues, KW-2189 and CB1-TM1), eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin γ1 and calicheamicin ω1; dynemicin, such as dynemicin A; uncialamycin and derivatives thereof; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-l-norleucine, doxorubicin (e.g., morpholino-doxorubicin, cyanomorpholino-doxorubicin, 2-pyrrolino-doxorubicin, or deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, or zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2″-trichloro-triethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”), cyclophosphamide; thiotepa; taxoids, e.g., paclitaxel and docetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; mitoxantrone; teniposide; edatrexate; daunomycin; aminopterin; capecitabine; ibandronate; irinotecan (e.g., CPT-11); topoisomerase inhibitor RFS 2000; difluorometlhylornithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosourea, dactinomycin, daunorubicin, doxorubicin, bleomycin, plicomycin, mitomycin, etoposide (VP16), tamoxifen, raloxifene, estrogen receptor binding agents, paclitaxel, docetaxel, gemcitabine, vinorelbine, farnesyl-protein transferase inhibitors, transplatinum, 5-fluorouracil, vincristine, vinblastine, or methotrexate or pharmaceutically acceptable salts, acids or derivatives of any of the above. Other examples of chemotherapeutic agents can be Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alkeran (Melphalan Hydrochloride), Alkeran (Melphalan), Alimta (Pemetrexed Disodium), Aloxi (Palonosetron Hydrochloride), Ambochlorin/Amboclorin (Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar (Irinotecan Hydrochloride), Capecitabine, CAPDX, Carac (Fluorouracil-Topical), Carboplatin, Carboplatin-Taxol, Carfilzomib, Carmubris (Carmustine), Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, Chlorambucil-prednisone, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Efudex (Fluorouracil-Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista (Raloxifene Hydrochloride), Evomela (Melphalan Hydrochloride), Exemestane, 5-FU (Fluorouracil Injection), 5-FU (Fluorouracil-Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil-Topical), Fluorouracil Injection, Fluorouracil-Topical, Flutamide, Folex (Methotrexate), Folex PFS (Methotrexate), FOLFIRI, FOLFIRI-bevacizumab, FOLFIRI-Cetuximab, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, Gemcitabine-Cisplatin, Gemcitabine-Oxaliplatin, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Ifex (Ifosfamide), Ifosfamide, Ifosfamidum (Ifosfamide), IL-2 (Aldesleukin), Imatinib Mesylate, Imbruvica (Ibrutinib), Imiquimod, Imlygic (Talimogene Laherparepvec), Inlyta (Axitinib), Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituximab, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq (Atezolizumab), Temodar (Temozolomide), Temozolomide, Temsirolimus, Thalidomide, Thalomid (Thalidomide), Thioguanine, Thiotepa, Tolak (Fluorouracil-Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zelboraf (Vemurafenib), Zevalin (Ibritumomab Tiuxetan), Zinecard (Dexrazoxane Hydrochloride), Ziv-Aflibercept, Zoladex (Goserelin Acetate), Zoledronic Acid, Zolinza (Vorinostat), Zometa (Zoledronic Acid), Zydelig (Idelalisib), Zykadia (Ceritinib), or Zytiga (Abiraterone Acetate) or pharmaceutically acceptable salts, acids or derivatives of any of the above.

Immunotherapy

As described herein, the provided methods allow for the identification of subjects who can benefit from immunotherapies. Immunotherapies are a new generation of cancer therapy that has revolutionized the treatment of otherwise terminal cancers, often achieving durable, sustained remission in cancers that were otherwise thought to be refractory to standard first- and second-line therapies. Thousands of patients annually are now treated with these life-saving therapies.

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

Examples of immunotherapy can be immune effector cell (IEC) therapy (e.g., CAR T, mesenchymal stem cells) or T cell engaging therapy (e.g., CD19-specific T cell engager, such as blinatumomab, T cell engaging monoclonal antibody, bispecific T cell engager (BiTE) therapy).

In some embodiments, the provided methods are used before, after, or in concurrence with any form of bispecific monoclonal antibody (BsMAb) therapy. For example, the BsMAb therapy can be any one or more of the currently FDA-approved BsMAb therapies, such as blinatumomab, emicizumab, or amivantamab.

In one aspect of immunotherapy, the tumor cell must bear some marker that is amenable to targeting, i.e., is not present on the majority of other cells. Many tumor markers exist and any of these may be suitable for targeting in the context of the present disclosure. Markers described herein are those in TABLE 3 and TABLE 9. Other common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, γ-IFN, chemokines such as MIP-1, MCP-1, IL-8, and growth factors such as FLT3 ligand. Combining immune stimulating molecules, either as proteins or using gene delivery in combination with a tumor suppressor has been shown to enhance anti-tumor effects (Ju et al., 2000). Moreover, antibodies against any of these compounds may be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides, et al., 1998), cytokine therapy, e.g., interferons α, β, and γ; IL-1, GM-CSF, TNF (Bukowski, et al., 1998; Davidson, et al., 1998; Hellstrand, et al., 1998) gene therapy, e.g., TNF, IL-1, IL-2, p53 (Qin et al., 1998; Austin-Ward and Villaseca, 1998; U.S. Pat. Nos. 5,830,880 and 5,846,945), and monoclonal antibodies, e.g., anti-ganglioside GM2, anti-HER-2, anti-p185 (Pietras, et al., 1998; Hanibuchi, et al., 1998; U.S. Pat. No. 5,824,311). It is contemplated that one or more anti-cancer therapies may be employed with the gene silencing therapies described herein.

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton, et al., 1992; Mitchell, et al., 1990; Mitchell, et al., 1993).

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg, et al., 1988; 1989).

In some embodiments, the immunotherapy in accordance with the present disclosure is CAR T cell therapy (e.g., CD19-specific chimeric antigen receptor T (CAR-T)). Generally, CAR T cell therapy refers to any type of immunotherapy in which a subject's T cells are genetically modified to express chimeric antigen receptors. These chimeric antigen receptors allow the T cells to more effectively recognize and subsequently destroy cancer cells. Typically, T cells are first harvested from a subject, genetically altered to express a CAR targeting an antigen of interest (e.g., an antigen expressed on the surface of a tumor or cancer cell), and then infused back into the subject. Once infused into the subject, CAR T cells bind to the target antigen and are activated, allowing them to proliferate and become cytotoxic.

Checkpoint Immunotherapy

In some embodiments, the present disclosure provides for a prediction of checkpoint immunotherapy response. An important function of the immune system is its ability to tell between normal cells in the body and those it sees as “foreign.” This lets the immune system attack the foreign cells while leaving the normal cells alone. To do this, it uses “checkpoints.” Immune checkpoints are molecules on certain immune cells that need to be activated (or inactivated) to start an immune response.

Cancer cells can find ways to use these checkpoints to avoid being attacked by the immune system. But drugs that target these checkpoints hold a lot of promise as a cancer treatment. These drugs are called checkpoint inhibitors. Checkpoint inhibitors used to treat cancer do not work directly on the tumor at all. They only take the brakes off an immune response that has begun but has not yet been working at its full force.

Checkpoint immunotherapy has been extensively shown to unleash T cell effector functions to control tumors in many cancer patients. However, tumor cells can evade immunological elimination by recruiting myeloid cells that induce an immunosuppressive state. Recent high dimensional profiling studies have shown that tumor-infiltrating myeloid cells are considerably heterogeneous, and may include both immunostimulatory and immunosuppressive subsets, although they do not fit the M1/M2 paradigm. Thus, depletion of suppressive myeloid cells from tumors, blockade of their functions, or induction of myeloid cells with immunostimulatory properties may provide important approaches for improving immunotherapy strategies, perhaps in synergy with checkpoint blockade.

Any immune checkpoint inhibitor known in the art can be used. For example, a PD-1 inhibitor can be used. These drugs are typically administered IV (intravenously). PD-1 is a checkpoint protein on immune cells called T cells. It normally acts as a type of “off switch” that helps keep the T cells from attacking other cells in the body. It does this when it attaches to PD-L1, a protein on some normal (and cancer) cells. When PD-1 binds to PD-L1, it tells the T cell to leave the other cell alone. Some cancer cells have large amounts of PD-L1, which helps them hide from an immune attack.

Monoclonal antibodies that target either PD-1 or PD-L1 can block this binding and boost the immune response against cancer cells. These drugs have shown a great deal of promise in treating certain cancers.

Examples of drugs that target PD-1 can include: Pembrolizumab (Keytruda), Nivolumab (Opdivo), Atezolizumab, or Cemiplimab (Libtayo). These drugs have been shown to be helpful in treating several types of cancer, and new cancer types are being added as more studies show these drugs to be effective.

As another example, a PD-L1 inhibitor can be used. Examples of drugs that target PD-L1 can include: Atezolizumab (Tecentriq), Avelumab (Bavencio), or Durvalumab (Imfinzi). These drugs have also been shown to be helpful in treating different types of cancer, and are being studied for use against others.

CTLA-4 is another protein on some T cells that acts as a type of “off switch” to keep the immune system in check. For example, Ipilimumab (Yervoy) is a monoclonal antibody that attaches to CTLA-4 and reduces or blocks its function. This can boost the body's immune response against cancer cells. This drug can be used to treat melanoma of the skin and other cancers.

Radiotherapy

In some embodiments, the methods described herein can provide evidence that a radiotherapy may be administered to a patient. Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter can repair themselves and function properly.

Radiation therapy used according to the present disclosure may include, but is not limited to, the use of γ-rays, X-rays, and/or the directed delivery of radioisotopes to tumor cells. Other forms of DNA damaging factors are also contemplated such as microwaves and UV-irradiation. It is most likely that all of these factors induce a broad range of damage on DNA, on the precursors of DNA, on the replication and repair of DNA, and on the assembly and maintenance of chromosomes. Dosage ranges for X-rays range from daily doses of 12.9 to 51.6 mC/kg for prolonged periods of time (3 to 4 wk), to single doses of 0.516 to 1.55 C/kg. Dosage ranges for radioisotopes vary widely, and depend on the half-life of the isotope, the strength and type of radiation emitted, and the uptake by the neoplastic cells.

Radiotherapy may comprise the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells.

Conformal radiotherapy uses the same radiotherapy machine, a linear accelerator, as the normal radiotherapy treatment but metal blocks are placed in the path of the x-ray beam to alter its shape to match that of the cancer or tumor. This ensures that a higher radiation dose is given to the tumor. Healthy surrounding cells and nearby structures receive a lower dose of radiation, so the possibility of side effects is reduced. A device called a multi-leaf collimator has been developed and may be used as an alternative to the metal blocks. The multi-leaf collimator consists of a number of metal sheets which are fixed to the linear accelerator. Each layer can be adjusted so that the radiotherapy beams can be shaped to the treatment area without the need for metal blocks. Precise positioning of the radiotherapy machine is very important for conformal radiotherapy treatment and a special scanning machine may be used to check the position of internal organs at the beginning of each treatment.

High-resolution intensity modulated radiotherapy also uses a multi-leaf collimator. During this treatment, the layers of the multi-leaf collimator are moved while the treatment is being given. This method is likely to achieve even more precise shaping of the treatment beams and allows the dose of radiotherapy to be constant over the whole treatment area.

Although research studies have shown that conformal radiotherapy and intensity modulated radiotherapy may reduce the side effects of radiotherapy treatment, it is possible that shaping the treatment area so precisely could stop microscopic cancer cells just outside the treatment area being destroyed. This means that the risk of the cancer coming back in the future may be higher with these specialized radiotherapy techniques.

Scientists also are looking for ways to increase the effectiveness of radiation therapy. Two types of investigational drugs are being studied for their effect on cells undergoing radiation. Radiosensitizers make the tumor cells more likely to be damaged, and radioprotectors protect normal tissues from the effects of radiation. Hyperthermia, the use of heat, is also being studied for its effectiveness in sensitizing tissue to radiation.

Surgical Treatment

In some embodiments, the methods described herein may be used to determine whether a subject would benefit from a surgical treatment for a urinary tract-associated cancer. A surgical treatment as used herein is a surgery performed on a subject having or suspected of having a urinary tract-associated cancer, wherein the surgery is intended to remove or reduce the urinary tract-associated cancer.

The appropriate surgical treatment is dependent on the type of urinary tract-associated cancer harbored by the subject. For example, a cystectomy, radical cystectomy, or transurethral resection of bladder tumor (TURBT) may be performed on a subject having bladder cancer. As another example, a nephroureterectomy or segmental ureterectomy may be performed on a subject having upper urinary tract cancer (e.g., a cancer in the renal pelvis or ureter).

Active Surveillance

In some embodiments, the methods described herein may be used to determine whether a subject is eligible to receive active surveillance (e.g., “watchful waiting”). As used herein, active surveillance refers to a method of disease management wherein the subject does not receive a cancer treatment, but rather is closely monitored over time by a clinician. Active surveillance may be used to avoid entirely or reduce the administration of a cancer treatment, such as a surgical treatment, chemotherapy, or radiotherapy, which are associated with significant morbidity and may be high-risk for certain patients. The methods described herein are used to determine whether a subject does or does not have residual disease, for example, after receiving a chemotherapy; if the subject is determined to not have residual disease, the subject may be administered active surveillance rather than additional chemotherapy or a surgical treatment.

Therapeutic Methods

Also provided is a process of treating a urinary tract-associated cancer in a subject in need of a cancer treatment, such as chemotherapy, immunotherapy, or radiotherapy, or alternatively active surveillance.

Methods described herein are generally performed on a subject in need thereof. A subject in need of the therapeutic methods described herein can be a subject having, diagnosed with, suspected of having, or at risk for developing a urinary-tract associated cancer. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; the activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Shamel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or a physician.

Administration of a cancer treatment or active surveillance can occur as a single event or over a time course of treatment. For example, a cancer treatment or active surveillance can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to or before, concurrent with, or after conventional treatment modalities for cancer.

Machine Learning (ML)

As shown herein, methods are described that obtain urine samples from subjects having or suspected of having a urinary tract-associated cancer, extract cell-free DNA (cfDNA), detect a cfDNA-derived metric, and employ a machine learning model to determine whether a subject has or does not have residual disease. For example, a random forest model was utilized to predict disease status based on variant allele frequency (VAF), tumor fraction (TFx), or tumor mutational value (TMB), along with clinical variables. Kaplan-Meier (KM) analysis was performed to assess overall survival (OS) and progression-free survival (PFS).

As described herein, ensemble methods can be used. Ensemble methods are meta-algorithms that combine several machine learning techniques into one predictive model in order to decrease variance (bagging), bias (boosting), or improve predictions (stacking). As an example, ensemble methods can be divided into two groups: (1) sequential ensemble methods and (2) parallel ensemble methods. Sequential ensemble methods are where the base learners are generated sequentially (e.g., AdaBoost). The basic motivation of sequential methods is to exploit the dependence between the base learners. The overall performance can be boosted by weighing previously mislabeled examples with higher weight. Parallel ensemble methods are where the base learners are generated in parallel (e.g., Random Forest). The basic motivation of parallel methods is to exploit independence between the base learners since the error can be reduced dramatically by averaging. Most ensemble methods use a single base learning algorithm to produce homogeneous base learners, i.e., learners of the same type, leading to homogeneous ensembles.

Heterogeneous learners can also be utilized, i.e., learners of different types, leading to heterogeneous ensembles. In order for ensemble methods to be more accurate than any of its individual members, the base learners should be as accurate as possible and as diverse as possible.

Bagging (bootstrap aggregation) can be used in the methods described herein, as a way to reduce the variance of an estimate is to average together multiple estimates. Bagging uses bootstrap sampling to obtain the data subsets for training the base learners. For aggregating the outputs of base learners, bagging can use voting for classification and averaging for regression. A class of ensemble algorithms that can be used are forests of randomized trees. In random forests, each tree in the ensemble is built from a sample drawn with replacement (i.e., a bootstrap sample) from the training set. In addition, instead of using all the features, a random subset of features is selected, further randomizing the tree. As a result, the bias of the forest increases slightly, but due to the averaging of less correlated trees, its variance decreases, resulting in an overall better model.

Boosting methods can also be used in the disclosed methods. Boosting refers to a family of algorithms that are able to convert weak learners to strong learners. The main principle of boosting is to fit a sequence of weak learners—models that are only slightly better than random guessing, such as small decision trees—to weighted versions of the data. More weight is given to examples that were misclassified by earlier rounds.

The predictions can then be combined through a weighted majority vote (classification) or a weighted sum (regression) to produce the final prediction. The principal difference between boosting and the committee methods, such as bagging, is that base learners are trained in sequence on a weighted version of the data. A form of boosting that can be used is an algorithm called AdaBoost, which stands for adaptive boosting. Another method that can be used is gradient tree boosting, a generalization of boosting to arbitrary differentiable loss functions. It can be used for both regression and classification problems. Gradient Boosting builds the model in a sequential way.

Stacking can be used to improve accuracy. Stacking is an ensemble learning technique that combines multiple classification or regression models via a meta-classifier or a meta-regressor. The base level models are trained based on a complete training set, then the meta-model is trained on the outputs of the base level model as features. The base level often consists of different learning algorithms and therefore stacking ensembles are often heterogeneous.

Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc., may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently, affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency; improve taste of the product; or improve shelf life of the product.

Molecular Engineering

The following definitions and methods are provided to better define the present disclosure and to guide those of ordinary skill in the art in the practice of the present disclosure. Unless otherwise noted, terms are to be understood according to conventional usage by those of ordinary skill in the relevant art.

The terms “heterologous DNA sequence”, “exogenous DNA segment”, or “heterologous nucleic acid,” as used herein, each refers to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above-required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A. For example, the percent identity can be at least 80% or about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

Substitution refers to the replacement of one amino acid with another amino acid in a protein or the replacement of one nucleotide with another in DNA or RNA. Insertion refers to the insertion of one or more amino acids in a protein or the insertion of one or more nucleotides with another in DNA or RNA. Deletion refers to the deletion of one or more amino acids in a protein or the deletion of one or more nucleotides with another in DNA or RNA. Generally, substitutions, insertions, or deletions can be made at any position so long as the required activity is retained.

So-called conservative exchanges can be carried out in which the amino acid which is replaced has a similar property as the original amino acid, for example, the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine); hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6 (log₁₀[Na⁺])+0.41 (fraction G/C content)−0.63 (% formamide)−(600/l). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated in the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| Side Chain Characteristic | Amino Acid |
|---|---|
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | D E |

Conservative Substitutions III

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | Ile, Leu, Met, Phe, Ala |

Exemplary nucleic acids that may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to a gene chip, EDTA, a centrifuge, resin slurry, LiCl, sodium acetate, micro spin column, micro column, potassium acetate, ethanol, nuclease free water, Tris, dsDNA assay kit, or a fluorometer. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or another substrate, and/or may be supplied as an electronic-readable medium or video. Detailed instructions may not be physically associated with the kit; instead, a user may be directed to an Internet web site specified by the manufacturer or distributor of the kit.

A control sample or a reference sample as described herein can be a sample from a healthy subject or sample, a wild-type subject or sample, or from populations thereof. A reference value can be used in place of a control or reference sample, which was previously obtained from a healthy subject or a group of healthy subjects or a wild-type subject or sample. A control sample or a reference sample can also be a sample with a known amount of a detectable compound or a spiked sample.

The methods and algorithms of the invention may be enclosed in a controller or processor. Furthermore, methods and algorithms of the present invention, can be embodied as a computer-implemented method or methods for performing such computer-implemented method or methods, and can also be embodied in the form of a tangible or non-transitory computer-readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer program include, for example, floppy disks and diskettes, compact disk (CD)-ROMs (whether or not writeable), DVD digital disks, RAM and ROM memories, computer hard drives and back-up drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods. The method or methods may be implemented on a general purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Urine Cell-Free DNA Multi-Omics to Detect MRD and Predict Survival in Bladder Cancer Patients

This Example describes the use of combinatorial ultra-deep targeted next generation sequencing (NGS) and ultra-low-pass whole genome sequencing (ULP-WGS) to sensitively detect MRD in urine and predict survival after curative-intent radical cystectomy.

Circulating tumor DNA (ctDNA) sensitivity remains subpar for molecular residual disease (MRD) detection in bladder cancer patients. To remedy this problem, the biofluid most proximal to the disease, urine, was obtained and urine tumor DNA was analyzed in 74 localized bladder cancer patients. Ultra-low-pass whole genome sequencing (ULP-WGS) was integrated with urine cancer personalized profiling by deep sequencing (uCAPP-Seq) to achieve sensitive MRD detection and predict overall survival. Variant allele frequency, inferred tumor mutational burden, and copy number-derived tumor fraction levels in urine cell-free DNA (cfDNA) significantly predicted pathologic complete response status, far better than plasma ctDNA was able to. A random forest model incorporating these urine cfDNA-derived factors with leave-one-out cross-validation was 87% sensitive for predicting residual disease in reference to gold-standard surgical pathology. Both progression-free survival (HR=3.00, p=0.01) and overall survival (HR=4.81, p=0.009) were dramatically worse by Kaplan-Meier analysis for patients predicted by the model to have MRD, which was corroborated by Cox regression analysis. Additional survival analyses performed on muscle-invasive, neoadjuvant chemotherapy, and held-out validation subgroups corroborated these findings. In summary, urine samples from 74 patients with localized bladder cancer were profiled and urine cfDNA multi-omics was used to detect MRD sensitively and predict survival accurately.

### INTRODUCTION

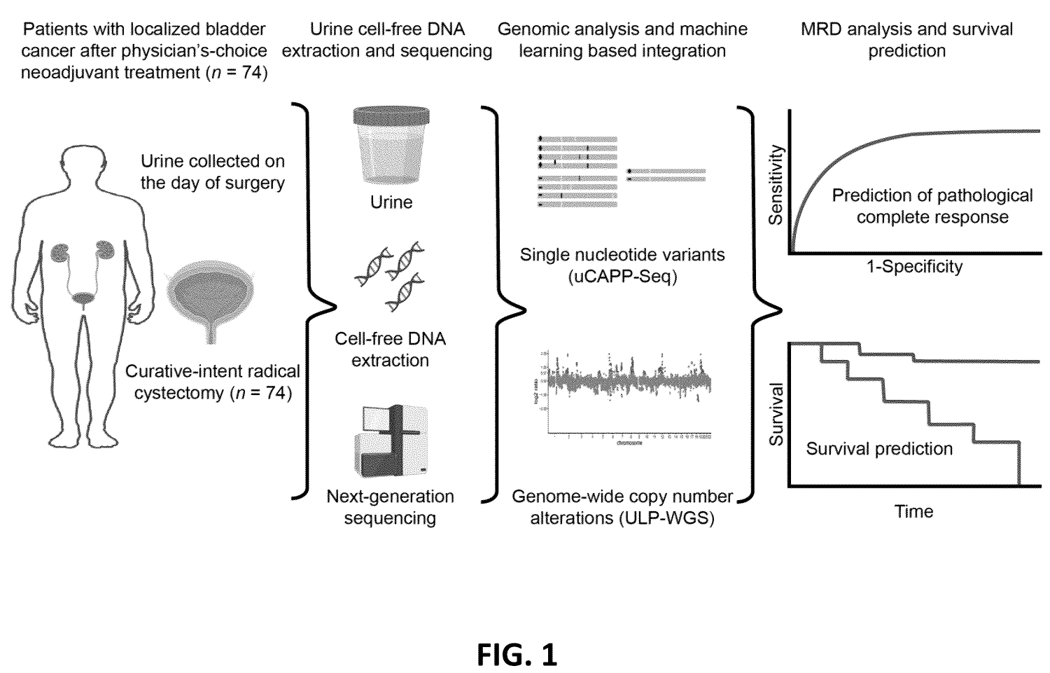

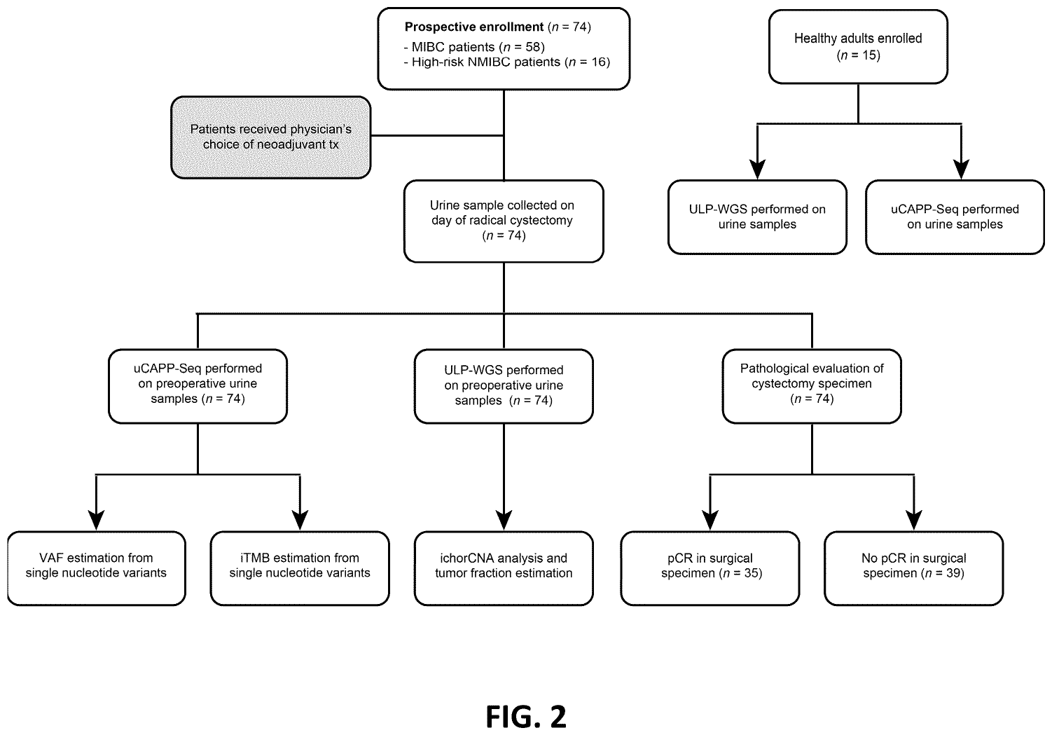

Bladder cancers shed tumor DNA into the urine, which can be measured using ultra-deep targeted sequencing. However, the modest sensitivity of this approach to detect molecular residual disease (MRD) limits clinical utility. Here, urine cell-free DNA (cfDNA) was analyzed using combinatorial ultra-deep targeted sequencing and ultra-low-pass whole genome sequencing (ULP-WGS) to sensitively detect MRD in urine and predict survival after curative-intent radical cystectomy (see e.g., FIG. 1 and FIG. 2).

Results

Cohort Characteristics and Biofluid Samples

Seventy-four localized bladder cancer patients underwent a physician's-choice of neoadjuvant treatment and curative-intent radical cystectomy. Seventy-eight percent (58/74) harbored muscle-invasive bladder cancer, while the rest had treatment-refractory non-muscle-invasive bladder cancer (see e.g., TABLE 1).

TABLE 1
Overview of the bladder cancer patient cohort.

| Patient characteristics (n = 74) | No. (%) | |
|---|---|---|
| Gender | | |
| Male | 58 | (78) |
| Female | 16 | (22) |
| Median Age (years) | 68 | |
| Median Follow-up (months) | 23 | |
| Ethnicity | | |
| White | 68 | (92) |
| Non-white | 6 | (8) |
| Smoking history | | |
| Yes | 51^{a} | (69) |
| No | 23 | (31) |
| T-stage (pre-treatment) | | |
| Ta | 2 | (3) |
| Tis | 2 | (3) |
| T1 | 12 | (16) |
| T2 | 52 | (70) |
| T3 | 6 | (8) |
| Neoadjuvant chemotherapy | | |
| Yes | 38 | (51) |
| ddMVAC | 9 | (12) |
| gemcitabine/cisplatin | 21 | (28) |
| gemcitabine/carboplatin | 1 | (1) |
| cisplatin/etoposide | 1 | (1) |
| carboplatin/paclitaxel | 1 | (1) |
| Treatment change^{b} | 1 | (1) |
| Unknown regimen | 4 | (5) |
| No | 36 | (49) |
| Pathologic complete response | 35 | (47) |
| Histology | | |
| Urothelial | 68 | (92) |
| Other | 6 | (8) |

^{a}Median 30 pack-years
^{b}Patient received 1 cycle of carboplatin/paclitaxel and 3 cycles of carboplatin/gemcitabine (switched due to cutaneous reaction to paclitaxel).

Ninety-two percent (68/74) had urothelial carcinoma, while the remainder had variant histologies. A full description of the cohort is displayed in TABLE 2.

TABLE 2
Patient-level characteristics.

| | Patient | | | | | Initial T |
|---|---|---|---|---|---|---|
| S.NO | ID | Age | Sex | Ethnicity | Histology | stageª |
| 1 | BC1045 | 63 | Male | White | urothelial carcinoma | T2 |
| 2 | BC1050 | 63 | Female | White | urothelial carcinoma | T2 |
| 3 | BC1058 | 78 | Male | White | urothelial carcinoma | T2 |
| 4 | BC1062 | 77 | Male | White | papillary urothelial | T2 |
| | | | | | carcinoma | |
| 5 | BC1063 | 67 | Male | White | urothelial carcinoma | T2 |
| 6 | BC1064 | 60 | Male | White | urothelial carcinoma | T2 |
| 7 | BC1065 | 64 | Male | White | urothelial carcinoma | T3 |
| 8 | BC1069 | 61 | Male | White | urothelial carcinoma, | T3 |
| | | | | | squamous | |
| | | | | | differentiation | |
| 9 | BC1071 | 68 | Male | White | urothelial carcinoma | T2 |
| 10 | BC1076 | 75 | Male | White | urothelial carcinoma, | at least |
| | | | | | trophoblastic | T2 |
| | | | | | differentiation | |
| 11 | BC1077 | 65 | Male | White | urothelial carcinoma | T2 |
| 12 | BC1083 | 66 | Male | White | urothelial carcinoma | T3 |
| 13 | BC1085 | 72 | Male | White | urothelial carcinoma | at least |
| | | | | | | T2 |
| 14 | BC1088 | 83 | Male | White | papillary urothelial | Ta |
| | | | | | carcinoma | |
| 15 | BC1090 | 83 | Male | White | urothelial carcinoma | T1 |
| 16 | BC1096 | 86 | Male | White | urothelial carcinoma | at least |
| | | | | | | T2 |
| 17 | BC1104 | 55 | Male | White | squamous cell | at least |
| | | | | | carcinoma | T2 |
| 18 | BC1105 | 68 | Male | White | urothelial carcinoma | T2 |
| 19 | BC1108 | 53 | Female | White | urothelial carcinoma | T1 |
| 20 | BC1109 | 83 | Male | White | urothelial carcinoma | Tis |
| 21 | BC1111 | 68 | Male | White | urothelial carcinoma | T1 |
| 22 | BC1116 | 72 | Female | Non- | papillary urothelial | T2 |
| | | | | White | carcinoma | |
| 23 | BC1132 | 51 | Male | White | urothelial carcinoma | T3 |
| 24 | BC1133 | 68 | Female | White | papillary urothelial | T2 |
| | | | | | carcinoma | |
| 25 | BC1135 | 59 | Female | White | urothelial carcinoma | T2 |
| 26 | BC1140 | 70 | Female | White | papillary urothelial | T1 |
| | | | | | carcinoma | |
| 27 | BC1147 | 71 | Male | White | urothelial carcinoma | at least |
| | | | | | | T2 |
| 28 | BC1171 | 79 | Male | Non- | urothelial carcinoma | T1 |
| | | | | White | | |
| 29 | BC1174 | 69 | Male | White | urothelial carcinoma | T1 |
| 30 | BC1182 | 72 | Male | White | urothelial carcinoma | T2 |
| 31 | BC1185 | 65 | Male | White | urothelial carcinoma | T2 |
| 32 | BC1186 | 82 | Female | White | urothelial carcinoma | T2 |
| 33 | BC1188 | 72 | Male | White | urothelial carcinoma | T2 |
| 34 | BC1196 | 60 | Male | White | urothelial carcinoma | at least |
| | | | | | | T2 |
| 35 | BC1197 | 64 | Male | White | urothelial carcinoma | at least |
| | | | | | | T2 |
| 36 | BC1198 | 63 | Female | White | urothelial carcinoma, | at least |
| | | | | | squamous | T2 |
| | | | | | differentiation | |
| 37 | BC1202 | 66 | Female | White | urothelial carcinoma | at least |
| | | | | | | T2 |
| 38 | BC1203 | 82 | Male | White | papillary urothelial | T2 |
| | | | | | carcinoma | |
| 39 | BC1205 | 64 | Male | White | urothelial carcinoma | T3 |
| 40 | BC1206 | 65 | Female | White | squamous cell | T3 |
| | | | | | carcinoma | |
| 41 | BC1207 | 76 | Male | White | urothelial carcinoma | T2 |
| 42 | BC1209 | 50 | Male | White | squamous cell | T2 |
| | | | | | carcinoma | |
| 51 | BC1215 | 76 | Male | White | urothelial carcinoma | Ta |
| 43 | BC1222 | 53 | Male | Black | urothelial carcinoma, | T2 |
| | | | | | squamous | |
| | | | | | differentiation | |
| 45 | BC1229 | 52 | Male | White | urothelial carcinoma | T2 |
| 44 | BC1233 | 74 | Male | White | urothelial carcinoma, | T2 |
| | | | | | squamous | |
| | | | | | differentiation | |
| 49 | BC1235 | 69 | Male | White | urothelial carcinoma | T2 |
| 46 | BC1242 | 48 | Female | White | papillary urothelial | T2 |
| | | | | | carcinoma | |
| 47 | BC1243 | 77 | Male | White | urothelial carcinoma | T2 |
| 54 | BC1255 | 69 | Male | White | urothelial carcinoma | T1 |
| 48 | BC1256 | 78 | Male | White | urothelial carcinoma | T2 |
| 50 | BC1260 | 84 | Male | White | urothelial carcinoma | T1 |
| 55 | BC1265 | 65 | Male | Black | urothelial carcinoma | T2 |
| 52 | BC1269 | 57 | Male | White | urothelial carcinoma | T2 |
| 56 | BC1274 | 62 | Male | White | urothelial carcinoma | T2 |
| 53 | BC1275 | 67 | Female | White | urothelial carcinoma | T1 |
| 57 | BC1277 | 76 | Female | White | urothelial carcinoma | T2 |
| 58 | BC1282 | 56 | Male | White | urothelial carcinoma | T2 |
| 59 | BC1284 | 75 | Male | White | urothelial carcinoma | Tis |
| 60 | BC1291 | 49 | Female | Asian | squamous cell | T2 |
| | | | | | carcinoma | |
| 61 | BC1295 | 65 | Male | White | urothelial carcinoma | T2 |
| 62 | BC1296 | 66 | Male | White | urothelial carcinoma | T2 |
| 63 | BC1298 | 50 | Male | White | urothelial carcinoma | T2 |
| 64 | BC1300 | 71 | Male | White | papillary urothelial | T2 |
| | | | | | carcinoma | |
| 65 | BC1303 | 76 | Male | White | urothelial carcinoma | T2 |
| 66 | BC1304 | 69 | Male | White | urothelial carcinoma | T2 |
| 67 | BC1308 | 78 | Female | White | urothelial carcinoma | T1 |
| 68 | BC1309 | 56 | Male | White | urothelial carcinoma | T1 |
| 69 | BC1310 | 50 | Male | White | urothelial carcinoma | T2 |
| 70 | BC1312 | 61 | Male | White | urothelial carcinoma | T1 |
| 71 | BC1313 | 48 | Male | White | squamous cell | T2 |
| | | | | | carcinoma | |
| 72 | BC1314 | 73 | Female | White | urothelial carcinoma | T2 |
| 73 | BC1315 | 76 | Male | Black | urothelial carcinoma | T2 |
| 74 | BC1316 | 60 | Male | White | neuroendocrine | T2 |

| | | | | Pathologic | |
|---|---|---|---|---|---|
| | De | Neoadjuvant | AJCC 8 Stage | complete | Smoking |
| S.NO | novo^{b} | chemotherapy | (pathologic)^{c} | response^{d} | History |
| 1 | Yes | Yes | ypT0N2 | No | Yes |
| 2 | Yes | Yes | ypT0N0 | Yes | Yes |
| 3 | Yes | No | PT1N0 | No | No |
| 4 | Yes | Yes | ypT2aN0 | No | Yes |
| 5 | Yes | Yes | ypT0N0 | Yes | No |
| 6 | Yes | Yes | ypTisN0 | Yes | Yes |
| 7 | No | Yes | ypTaN0 | Yes | No |
| 8 | Yes | No | pT3aN2 | No | Yes |
| 9 | Yes | No | pT4aN2 | No | No |
| 10 | Yes | No | pT3aN0 | No | Yes |
| 11 | Yes | Yes | ypT3aN0 | No | Yes |
| 12 | Yes | Yes | ypT3aN0 | No | Yes |
| 13 | No | No | pT4aN1 | No | Yes |
| 14 | No | No | PT1N0 | No | No |
| 15 | No | No | pT3aN0 | No | No |
| 16 | Yes | No | pT3aN2 | No | No |
| 17 | Yes | Yes | ypT2bN0 | No | No |
| 18 | Yes | Yes | ypT1N0 | No | Yes |
| 19 | No | No | pTisN0 | Yes | No |
| 20 | No | No | pTisN0 | Yes | Yes |
| 21 | No | No | pT0N0 | Yes | Yes |
| 22 | No | No | pT2bN1 | No | Yes |
| 23 | No | Yes | ypT3bN2 | No | No |
| 24 | No | No | pTaN0 | Yes | Yes |
| 25 | No | Yes | ypT3aN1 | No | Yes |
| 26 | No | No | PT1N0 | No | Yes |
| 27 | Yes | No | pT3aN1 | No | No |
| 28 | Yes | No | pT3aN0 | No | Yes |
| 29 | No | No | pTisN0 | Yes | Yes |
| 30 | Yes | No | pT3aN2 | No | Yes |
| 31 | Yes | Yes | ypTisN0 | Yes | Yes |
| 32 | Yes | No | pT3aN0 | No | No |
| 33 | No | No | pT2aN0 | No | Yes |
| 34 | Yes | Yes | ypT0N0 | Yes | Yes |
| 35 | Yes | Yes | ypT0N0 | Yes | Yes |
| 36 | Yes | Yes | ypT0N1 | Yes | Yes |
| 37 | No | No | pT3aN0 | No | Yes |
| 38 | Yes | Yes | ypTaN0 | Yes | Yes |
| 39 | Yes | Yes | ypTisN0 | Yes | Yes |
| 40 | Yes | No | pT4bN1Mx | No | No |
| 41 | Yes | Yes | ypT0N0Mx | Yes | Yes |
| 42 | Yes | No | pT3aN0Mx | No | No |
| 51 | No | No | pT0N0 | Yes | Yes |
| 43 | No | No | pT2bN1Mx | No | Yes |
| 45 | No | Yes | ypT3bN2Mx | No | Yes |
| 44 | Yes | No | pT3bN1Mx | No | Yes |
| 49 | Yes | Yes | ypT1M0 | No | Yes |
| 46 | Yes | Yes | ypT0N0 | Yes | Yes |
| 47 | No | Yes | ypTisN0M0 | Yes | Yes |
| 54 | Yes | No | pTaN0 | Yes | Yes |
| 48 | Yes | Yes | ypT0N0 | Yes | Yes |
| 50 | No | No | pT4aN0 | No | No |
| 55 | Yes | Yes | ypTisN0 | Yes | Yes |
| 52 | Yes | Yes | ypT4bN0 | No | No |
| 56 | Yes | Yes | ypT0N0 | Yes | Yes |
| 53 | No | No | PT1N1 | No | Yes |
| 57 | Yes | Yes | ypT0N0 | Yes | Yes |
| 58 | Yes | Yes | ypT0N0 | Yes | No |
| 59 | No | No | pTisN0 | Yes | Yes |
| 60 | Yes | No | pT3aN2 | No | Yes |
| 61 | Yes | Yes | ypT0N0 | Yes | Yes |
| 62 | Yes | Yes | ypT0N0 | Yes | Yes |
| 63 | Yes | Yes | ypT0N0 | Yes | Yes |
| 64 | Yes | No | pT4aN1 | No | Yes |
| 65 | Yes | No | pT0N0 | Yes | No |
| 66 | Yes | No | pT3aN0 | No | Yes |
| 67 | Yes | No | pT0N0 | Yes | Yes |
| 68 | No | No | pT0N0 | Yes | Yes |
| 69 | No | Yes | ypT4N0Mx | No | No |
| 70 | No | Yes | ypT0N0 | Yes | No |
| 71 | Yes | Yes | ypT4aN2 | No | No |
| 72 | Yes | Yes | ypT0N0 | Yes | Yes |
| 73 | No | Yes | ypT3aN2 | No | No |
| 74 | Yes | Yes | ypTisN0 | Yes | No |

| | | | | | SNV- | |
|---|---|---|---|---|---|---|
| | | | | | derived | inferred |
| | | Body | | | variant | Tumor |
| | | mass | | Urine | allele | mutational |
| | Pack- | index | Hematocrit | cfDNA | frequency^{e} | burden |
| S. NO | Years | (kg/m2) | (%) | (ng/ml) | (%) | (iTMB)^{f} |
| 1 | 45 | 25.5 | 34.0 | 108.2 | 2.4 | 340 |
| 2 | 40 | 27.6 | 29.0 | 15.1 | 0.0 | 74 |
| 3 | 0 | 31.7 | 43.0 | 8.2 | 5.9 | 136 |
| 4 | 20 | 21.4 | 33.0 | 3.9 | 2.4 | 170 |
| 5 | 0 | 25.5 | 36.8 | 46.8 | 0.0 | 40 |
| 6 | 2 | 32.6 | 34.0 | 24.9 | 0.0 | 68 |
| 7 | 0 | 27.8 | 30.0 | 136.5 | 0.0 | 137 |
| 8 | 30 | 29.7 | 22.0 | 13.0 | 11.6 | 204 |
| 9 | 0 | 34.5 | 28.0 | 47.5 | 10.1 | 170 |
| 10 | 25 | 28.3 | 27.0 | 131.3 | 3.8 | 306 |
| 11 | 30 | 28.2 | 40.0 | 3.4 | 9.6 | 170 |
| 12 | 22 | 26.9 | 35.0 | 155.5 | 5.6 | 476 |
| 13 | 56 | 26.0 | 35.0 | 98.2 | 52.2 | 272 |
| 14 | 0 | 26.7 | 50.8 | 40.6 | 7.4 | 170 |
| 15 | 0 | 31.6 | 34.0 | 76.8 | 3.5 | 204 |
| 16 | 0 | 26.3 | 34.0 | 36.5 | 74.3 | 272 |
| 17 | 0 | 27.6 | 34.7 | 68.3 | 2.4 | 111 |
| 18 | 20 | 31.3 | 32.8 | 1.0 | 0.4 | 142 |
| 19 | 0 | 24.5 | 34.0 | 34.8 | 0.0 | 340 |
| 20 | 15 | 26.8 | 41.0 | 23.2 | 0.0 | 171 |
| 21 | 30 | 29.1 | 40.0 | 21.0 | 7.3 | 204 |
| 22 | 30 | 21.3 | 36.0 | 75.8 | 0.0 | 34 |
| 23 | 0 | 30.6 | 39.0 | 20.6 | 0.0 | 69 |
| 24 | 10.2 | 25.5 | 37.0 | 24.5 | 12.5 | 122 |
| 25 | 30 | 21.8 | 24.0 | 3.5 | 4.8 | 136 |
| 26 | 5.5 | 20.5 | 38.0 | 72.4 | 0.0 | 34 |
| 27 | 0 | 17.8 | 45.0 | 19.5 | 0.0 | 116 |
| 28 | 45 | 31.1 | 33.0 | 220.4 | 95.2 | 204 |
| 29 | 40 | 39.2 | 39.0 | 16.7 | 11.8 | 204 |
| 30 | 30 | 29.3 | 35.0 | 93.3 | 19.9 | 170 |
| 31 | 0.75 | 21.7 | 34.0 | 8.5 | 1.7 | 102 |
| 32 | 0 | 19.0 | 24.0 | 391.2 | 3.4 | 238 |
| 33 | 20 | 25.4 | 45.0 | 25.9 | 21.0 | 272 |
| 34 | 45 | 20.0 | 38.0 | 4.7 | 0.0 | 34 |
| 35 | 42 | 32.2 | 30.0 | 25.2 | 0.0 | 117 |
| 36 | 38 | 23.8 | 33.0 | 20.9 | 0.0 | 112 |
| 37 | 30 | 30.3 | 37.9 | 4.8 | 2.4 | 206 |
| 38 | 42 | 29.3 | 33.0 | 48.0 | 0.0 | 93 |
| 39 | 50 | 31.8 | 34.0 | 7.9 | 0.7 | 75 |
| 40 | 0 | 17.9 | 28.0 | 240.0 | 2.8 | 170 |
| 41 | 30 | 26.0 | 41.0 | 2.2 | 0.0 | 0 |
| 42 | 0 | 27.1 | 33.0 | 270.0 | 7.3 | 204 |
| 51 | 25 | 32.6 | 31.0 | 118.8 | 0.0 | 69 |
| 43 | Not | 25.0 | 44.0 | 672.0 | 0.0 | 442 |
| | specified | | | | | |
| 45 | 28 | 23.4 | 29.8 | 11.9 | 5.6 | 102 |
| 44 | 20 | 27.0 | 38.6 | 164.0 | 0.0 | 204 |
| 49 | 8 | 28.3 | 38.3 | 0.5 | 0.0 | 76 |
| 46 | 15 | 29.4 | 36.0 | 60.1 | 0.6 | 105 |
| 47 | 5 | 28.7 | 30.1 | 93.2 | 0.0 | 210 |
| 54 | 100 | 41.6 | 39.0 | 16.6 | 53.4 | 170 |
| 48 | 40 | 22.4 | 30.9 | 4.7 | 0.0 | 40 |
| 50 | 0 | 24.1 | 42.0 | 12.1 | 0.8 | 238 |
| 55 | 45 | 21.6 | 35.0 | 10.8 | 7.3 | 170 |
| 52 | 0 | 19.6 | 30.8 | 13.3 | 4.7 | 204 |
| 56 | 5 | 26.2 | 38.0 | 1.7 | 13.0 | 204 |
| 53 | 20 | 27.9 | 31.4 | 3.8 | 8.8 | 170 |
| 57 | 15 | 25.0 | 27.4 | 16.0 | 0.0 | 40 |
| 58 | 0 | 25.4 | 27.4 | 0.8 | 0.0 | 68 |
| 59 | 5 | 31.9 | 37.0 | 3.7 | 0.0 | 137 |
| 60 | 10 | 18.5 | 32.0 | 56.4 | 24.3 | 204 |
| 61 | 35 | 24.3 | 32.0 | 9.3 | 5.3 | 170 |
| 62 | 40 | 23.7 | 30.0 | 42.0 | 0.0 | 37 |
| 63 | 30 | 22.9 | 35.1 | 70.6 | 0.4 | 68 |
| 64 | 30 | 35.3 | 32.0 | 151.7 | 0.0 | 205 |
| 65 | 0 | 32.2 | 23.0 | 38.0 | 0.2 | 238 |
| 66 | 30 | 26.9 | 32.0 | 22.4 | 17.0 | 306 |
| 67 | 50 | 45.8 | 28.0 | 31.7 | 26.1 | 272 |
| 68 | 25 | 26.5 | 42.0 | 43.2 | 4.7 | 170 |
| 69 | 0 | 39.5 | 30.0 | 15.7 | 9.9 | 102 |
| 70 | 0 | 25.8 | 40.0 | 69.1 | 0.0 | 60 |
| 71 | 0 | 26.6 | 28.0 | 161.1 | 1.9 | 272 |
| 72 | 40 | 34.9 | 27.0 | 133.1 | 0.3 | 272 |
| 73 | 0 | 25.0 | 35.0 | 21.6 | 31.3 | 340 |
| 74 | 0 | 27.9 | 35.0 | 57.0 | 1.8 | 170 |
| | CNA- | | | | | |
| | inferred | LOOCV | | | Death | |
| | tumor | RF | Progression | | during | |
| | fraction^{g} | model | during | PFS | follow- | OS |
| S.NO | (%) | score^{h} | follow-up | (months)ⁱ | up | (months)ⁱ |
| 1 | 2.1 | 0.82 | Yes | 10.2 | No | 38.5 |
| 2 | 2.5 | 0.90 | No | 32.8 | No | 36.1 |
| 3 | 4.1 | 0.22 | No | 28.3 | No | 37.1 |
| 4 | 1.6 | 0.59 | No | 24.8 | No | 25.8 |
| 5 | 3.9 | 0.57 | Yes | 8.4 | No | 37.5 |
| 6 | 0.0 | 0.93 | No | 29.8 | No | 33.3 |
| 7 | 1.0 | 0.89 | No | 32.8 | No | 38.2 |
| 8 | 12.9 | 0.36 | No | 37.8 | No | 37.8 |
| 9 | 0.0 | 0.33 | Yes | 6.0 | Yes | 6.0 |
| 10 | 6.8 | 0.12 | No | 9.3 | No | 27.5 |
| 11 | 13.9 | 0.53 | No | 34.3 | No | 34.3 |
| 12 | 7.8 | 0.28 | Yes | 2.3 | Yes | 3.7 |
| 13 | 49.3 | 0.12 | Yes | 2.1 | Yes | 6.5 |
| 14 | 9.1 | 0.10 | No | 20.0 | No | 34.9 |
| 15 | 14.6 | 0.10 | No | 2.8 | No | 26.2 |
| 16 | 50.0 | 0.07 | Yes | 7.5 | Yes | 8.1 |
| 17 | 2.2 | 0.66 | No | 16.4 | No | 28.1 |
| 18 | 0.0 | 0.72 | No | 34.8 | No | 34.8 |
| 19 | 0.0 | 0.44 | No | 24.7 | No | 33.9 |
| 20 | 0.0 | 0.66 | No | 22.4 | No | 28.9 |
| 21 | 4.8 | 0.61 | No | 25.6 | No | 25.6 |
| 22 | 24.4 | 0.29 | No | 16.1 | No | 32.2 |
| 23 | 1.4 | 0.75 | Yes | 6.6 | No | 33.2 |
| 24 | 24.0 | 0.05 | No | 24.9 | No | 29.5 |
| 25 | 9.9 | 0.58 | No | 11.3 | No | 18.1 |
| 26 | 22.7 | 0.68 | Yes | 26.3 | No | 31.1 |
| 27 | 3.6 | 0.59 | Yes | 7.3 | Yes | 9.0 |
| 28 | 45.9 | 0.59 | No | 25.7 | No | 28.5 |
| 29 | 8.0 | 0.61 | Yes | 22.8 | No | 28.2 |
| 30 | 19.4 | 0.02 | No | 5.3 | No | 5.3 |
| 31 | 2.4 | 0.67 | Yes | 11.8 | Yes | 14.6 |
| 32 | 13.8 | 0.16 | No | 12.5 | No | 25.5 |
| 33 | 16.0 | 0.02 | Yes | 12.8 | No | 24.7 |
| 34 | 5.9 | 0.82 | No | 4.2 | No | 25.1 |
| 35 | 1.8 | 0.89 | No | 22.8 | No | 23.9 |
| 36 | 0.0 | 0.95 | No | 20.4 | No | 23.3 |
| 37 | 1.6 | 0.39 | No | 22.8 | No | 22.8 |
| 38 | 0.0 | 0.87 | No | 4.3 | No | 23.2 |
| 39 | 4.1 | 0.92 | No | 4.7 | No | 13.0 |
| 40 | 5.8 | 0.20 | Yes | 1.5 | Yes | 5.4 |
| 41 | 0.0 | 0.93 | No | 15.7 | No | 18.7 |
| 42 | 8.4 | 0.07 | Yes | 6.0 | Yes | 13.4 |
| 51 | 0.0 | 0.90 | No | 12.9 | No | 13.2 |
| 43 | 9.8 | 0.24 | No | 19.1 | No | 21.4 |
| 45 | 9.4 | 0.36 | Yes | 5.9 | No | 16.4 |
| 44 | 61.6 | 0.07 | Yes | 1.9 | Yes | 8.4 |
| 49 | 2.5 | 0.65 | Yes | 10.1 | No | 13.9 |
| 46 | 2.8 | 0.70 | No | 14.6 | No | 14.7 |
| 47 | 0.0 | 0.53 | No | 16.3 | No | 16.3 |
| 54 | 58.3 | 0.61 | No | 10.5 | No | 10.6 |
| 48 | 0.0 | 0.94 | No | 11.5 | No | 13.8 |
| 50 | 21.0 | 0.12 | Yes | 5.6 | No | 13.3 |
| 55 | 6.1 | 0.32 | Yes | 4.6 | No | 7.7 |
| 52 | 4.8 | 0.28 | Yes | 5.0 | Yes | 8.8 |
| 56 | 6.0 | 0.12 | No | 6.8 | No | 9.0 |
| 53 | 17.1 | 0.66 | No | 3.9 | No | 10.9 |
| 57 | 0.0 | 0.97 | No | 9.1 | No | 11.7 |
| 58 | 4.5 | 0.60 | Yes | 3.9 | Yes | 6.4 |
| 59 | 2.8 | 0.70 | No | 9.5 | No | 10.4 |
| 60 | 12.9 | 0.30 | Yes | 5.6 | Yes | 7.4 |
| 61 | 3.7 | 0.25 | No | 9.5 | No | 9.5 |
| 62 | 0.0 | 0.82 | No | 8.7 | No | 10.2 |
| 63 | 3.4 | 0.66 | No | 6.6 | No | 6.6 |
| 64 | 0.0 | 0.42 | No | 2.3 | No | 5.0 |
| 65 | 1.3 | 0.23 | No | 3.6 | No | 9.2 |
| 66 | 14.0 | 0.31 | No | 5.9 | No | 6.1 |
| 67 | 55.9 | 0.27 | No | 0.6 | No | 4.7 |
| 68 | 0.0 | 0.59 | No | 5.4 | No | 8.1 |
| 69 | 11.6 | 0.07 | Yes | 5.9 | No | 8.9 |
| 70 | 0.0 | 0.83 | No | 3.2 | No | 5.8 |
| 71 | 12.9 | 0.08 | Yes | 1.7 | No | 5.0 |
| 72 | 0.0 | 0.66 | No | 4.8 | No | 5.4 |
| 73 | 20.9 | 0.34 | No | 5.1 | No | 7.4 |
| 74 | 3.9 | 0.24 | No | 2.5 | No | 5.3 |

^{a}Staging was performed at the time of pre-treatment TURBT using AJCC 8 criteria.
^{b}De novo refers to whether this instance of localized bladder cancer was a new diagnosis.
^{c}Staging was performed at the time of radical cystectomy using AJCC 8 criteria, with “y” indicating the patient received neoadjuvant chemotherapy.
^{d}Pathologic complete response status was defined as T0, Ta or Tis in the radical cystectomy specimen.
^{e}SNV-derived maximum non-silent variant allele frequency (VAF) in urine cell-free DNA, determined by uCAPP-Seq.
^{f}Exome-wide iTMB interpolated from uCAPP-Seq results.
^{g}Copy number-inferred tumor fraction estimated from ULP-WGS of urine cell-free DNA.
^{h}LOOCV random forest model score for predicting pCR vs. MRD.
ⁱProgression-free survival (PFS) and overall survival (OS) were from the time of radical cystectomy.

Urine cancer personalized profiling by deep sequencing (uCAPP-Seq) libraries prepared from urine cfDNA samples were sequenced to >900× median unique depth (see e.g., TABLE 3) along with comparably sequenced plasma (see e.g., TABLE 4) and germline DNA (see e.g., TABLE 5).

TABLE 3
Sequencing metrics for urine cell-free DNA libraries analyzed by
CAPP-Seq.

| | | | | Median | Median | |
|---|---|---|---|---|---|---|
| | NGS | On-target | | unique | fragment | Duplex |
| Subject ID | reads^{a} | rate^{b} | Median depth^{c} | depth^{d} | length (bp)^{e} | rate^{f} |
| BC1045 | 86044908 | 66% | 12896 | 1672 | 211 | 14% |
| BC1050 | 112448596 | 45% | 12047 | 416 | 197 | 19% |
| BC1058 | 80082917 | 36% | 6964 | 803 | 199 | 22% |
| BC1062 | 67273563 | 63% | 10779 | 1426 | 195 | 20% |
| BC1063 | 59802599 | 59% | 6434 | 489 | 186 | 24% |
| BC1064 | 114778624 | 45% | 13737 | 487 | 194 | 19% |
| BC1065 | 39517627 | 36% | 3800 | 942 | 227 | 8% |
| BC1069 | 53041349 | 32% | 4562 | 1068 | 198 | 8% |
| BC1071 | 109814511 | 45% | 7547 | 1298 | 206 | 10% |
| BC1076 | 69313200 | 64% | 6921 | 492 | 218 | 11% |
| BC1077 | 84762979 | 66% | 12886 | 485 | 215 | 25% |
| BC1083 | 131505614 | 54% | 15722 | 1458 | 190 | 13% |
| BC1085 | 111290583 | 46% | 13793 | 811 | 220 | 10% |
| BC1088 | 51991502 | 37% | 4383 | 912 | 184 | 15% |
| BC1090 | 93625053 | 48% | 12061 | 558 | 223 | 17% |
| BC1096 | 165042082 | 44% | 13953 | 639 | 189 | 6% |
| BC1104 | 63724347 | 35% | 5805 | 483 | 198 | 29% |
| BC1105 | 77435537 | 66% | 11461 | 459 | 202 | 29% |
| BC1108 | 128660774 | 40% | 13423 | 900 | 197 | 4% |
| BC1109 | 114256260 | 42% | 11524 | 568 | 196 | 11% |
| BC1111 | 95443086 | 22% | 5256 | 806 | 194 | 24% |
| BC1116 | 137313246 | 45% | 14536 | 843 | 203 | 6% |
| BC1132 | 106717842 | 47% | 13667 | 753 | 221 | 15% |
| BC1133 | 53052535 | 35% | 4796 | 690 | 198 | 29% |
| BC1135 | 75073502 | 39% | 6987 | 426 | 224 | 30% |
| BC1140 | 51949517 | 62% | 8517 | 430 | 216 | 30% |
| BC1147 | 44255080 | 36% | 3878 | 776 | 193 | 19% |
| BC1171 | 67617578 | 68% | 9606 | 1487 | 205 | 16% |
| BC1174 | 74419432 | 38% | 7301 | 1083 | 225 | 23% |
| BC1182 | 60706405 | 67% | 8902 | 684 | 200 | 30% |
| BC1185 | 73723253 | 46% | 8165 | 1027 | 230 | 25% |
| BC1186 | 59438216 | 45% | 6491 | 989 | 230 | 18% |
| BC1188 | 40536268 | 40% | 3803 | 761 | 189 | 20% |
| BC1196 | 53238995 | 22% | 3077 | 828 | 205 | 20% |
| BC1197 | 50491235 | 23% | 3165 | 499 | 191 | 31% |
| BC1198 | 63807796 | 62% | 9937 | 1496 | 208 | 23% |
| BC1202 | 53258914 | 60% | 7863 | 758 | 211 | 26% |
| BC1203 | 62150154 | 15% | 2346 | 714 | 195 | 15% |
| BC1205 | 52853500 | 23% | 3091 | 834 | 201 | 22% |
| BC1206 | 76458550 | 60% | 10843 | 1665 | 187 | 15% |
| BC1207 | 43917622 | 16% | 1832 | 460 | 198 | 21% |
| BC1209 | 61907852 | 15% | 2285 | 830 | 191 | 6% |
| BC1215 | 41840817 | 66% | 6898 | 1965 | 199 | 11% |
| BC1222 | 45514847 | 73% | 8000 | 1070 | 183 | 17% |
| BC1229 | 26932061 | 62% | 4180 | 508 | 211 | 28% |
| BC1233 | 56810894 | 65% | 9969 | 1281 | 173 | 17% |
| BC1235 | 41678631 | 63% | 6261 | 1238 | 208 | 16% |
| BC1242 | 36792104 | 70% | 6429 | 1378 | 203 | 17% |
| BC1243 | 38226312 | 69% | 6272 | 1453 | 195 | 13% |
| BC1255 | 44393566 | 67% | 6886 | 1112 | 189 | 20% |
| BC1256 | 35072133 | 66% | 5337 | 654 | 190 | 25% |
| BC1260 | 46267094 | 64% | 6940 | 1503 | 199 | 15% |
| BC1265 | 25508923 | 47% | 3135 | 835 | 221 | 11% |
| BC1269 | 41237934 | 69% | 6242 | 942 | 184 | 22% |
| BC1274 | 35732216 | 62% | 5136 | 465 | 195 | 30% |
| BC1275 | 30525125 | 62% | 4710 | 244 | 200 | 28% |
| BC1277 | 30662462 | 63% | 4842 | 495 | 203 | 31% |
| BC1282 | 53045283 | 70% | 9817 | 1014 | 279 | 18% |
| BC1284 | 61760887 | 73% | 11375 | 1995 | 206 | 16% |
| BC1291 | 60915211 | 69% | 9862 | 1589 | 183 | 13% |
| BC1295 | 42146652 | 76% | 8219 | 1055 | 234 | 17% |
| BC1296 | 66106735 | 75% | 12555 | 2058 | 212 | 11% |
| BC1298 | 55358238 | 71% | 9812 | 1859 | 203 | 12% |
| BC1300 | 76328311 | 73% | 14044 | 2547 | 186 | 12% |
| BC1303 | 88821595 | 77% | 17058 | 2483 | 214 | 15% |
| BC1304 | 63237968 | 78% | 11939 | 1826 | 190 | 14% |
| BC1308 | 44982582 | 62% | 7339 | 2009 | 225 | 8% |
| BC1309 | 35853566 | 61% | 5744 | 1545 | 230 | 10% |
| BC1310 | 40353507 | 61% | 6404 | 1813 | 215 | 8% |
| BC1312 | 42368243 | 65% | 6749 | 1611 | 187 | 7% |
| BC1313 | 56422074 | 60% | 8078 | 1993 | 191 | 7% |
| BC1314 | 53877425 | 60% | 7507 | 1766 | 213 | 8% |
| BC1315 | 52367166 | 62% | 6803 | 1290 | 195 | 11% |
| BC1316 | 33797273 | 62% | 4813 | 1079 | 189 | 6% |

^{a}Number of total sequencing reads per sample after demultiplexing.
^{b}Percentage of quality-control-passed reads overlapping with the targeted hybrid-capture panel.
^{c}Median non-deduplicated sequencing depth.
^{d}Median UMI-deduplicated sequencing depth.
^{e}Median length of sonicated urine cell-free DNA fragments.
^{f}Percentage of UMI-deduplicated reads with duplex support.

TABLE 4
Sequencing metrics for plasma cell-free
DNA libraries analyzed by CAPP-Seq.

| | | | | | Median |
|---|---|---|---|---|---|
| | | | | Median | fragment |
| | NGS | On-target | Median | unique | length |
| Subject ID | reads^{a} | rate^{b} | depth^{c} | depth^{d} | (bp)^{e} |
| BC1045 | 42627513 | 48.9% | 5237 | 536 | 178 |
| BC1050 | 55403148 | 60.8% | 8151 | 702 | 198 |
| BC1058 | 53348089 | 47.5% | 6668 | 614 | 204 |
| BC1062 | 61125503 | 52.1% | 8230 | 1827 | 180 |
| BC1063 | 45125980 | 47.9% | 5669 | 564 | 206 |
| BC1064 | 61090860 | 60.0% | 8848 | 673 | 185 |
| BC1065 | 56821148 | 48.5% | 7173 | 677 | 195 |
| BC1069 | 53774675 | 45.2% | 6086 | 574 | 186 |
| BC1071 | 55355592 | 62.1% | 8655 | 703 | 185 |
| BC1076 | 58302353 | 60.5% | 8859 | 724 | 196 |
| BC1077 | 57649656 | 48.5% | 7223 | 705 | 189 |
| BC1083 | 57812239 | 61.1% | 8498 | 669 | 177 |
| BC1085 | 59137684 | 58.7% | 8573 | 627 | 191 |
| BC1088 | 83738811 | 24.3% | 5580 | 1164 | 203 |
| BC1090 | 51680361 | 59.0% | 7400 | 629 | 282 |
| BC1096 | 60599276 | 59.7% | 8636 | 675 | 178 |
| BC1104 | 74992390 | 24.3% | 5088 | 1275 | 198 |
| BC1105 | 45435410 | 48.4% | 5561 | 528 | 200 |
| BC1108 | 57373441 | 57.1% | 7848 | 641 | 179 |
| BC1109 | 57786890 | 59.4% | 7751 | 633 | 175 |
| BC1111 | 84598577 | 23.3% | 5415 | 1267 | 187 |
| BC1116 | 58519908 | 58.8% | 8469 | 654 | 190 |
| BC1132 | 63178526 | 57.8% | 8472 | 662 | 183 |
| BC1133 | 75210147 | 22.6% | 4711 | 1112 | 205 |
| BC1135 | 51514185 | 47.2% | 6377 | 560 | 209 |
| BC1140 | 90833579 | 22.9% | 5543 | 1135 | 184 |
| BC1147 | 76191798 | 23.3% | 4833 | 984 | 267 |
| BC1171 | 52038449 | 48.8% | 6467 | 642 | 183 |
| BC1174 | 86454806 | 23.2% | 5431 | 1259 | 188 |
| BC1182 | 58621955 | 49.0% | 7530 | 710 | 197 |
| BC1185 | 48136974 | 45.7% | 5823 | 565 | 272 |
| BC1186 | 49511953 | 47.6% | 6041 | 593 | 214 |
| BC1188 | 88619000 | 22.4% | 4264 | 1062 | 172 |
| BC1197 | 42885042 | 63.5% | 6869 | 1958 | 177 |
| BC1202 | 57300054 | 48.8% | 7456 | 2554 | 182 |
| BC1203 | 62931913 | 49.7% | 7117 | 2605 | 172 |
| BC1205 | 48238196 | 49.6% | 6540 | 2411 | 192 |
| BC1206 | 89559763 | 47.2% | 11136 | 3227 | 180 |
| BC1207 | 73548473 | 45.4% | 8865 | 2634 | 191 |
| BC1209 | 67975395 | 47.3% | 8599 | 2575 | 195 |

^{a}Number of total sequencing reads per sample after demultiplexing.
^{b}Percentage of quality-control-passed reads overlapping with the targeted hybrid-capture panel.
^{c}Median non-deduplicated sequencing depth.
^{d}Median UMI-deduplicated sequencing depth.
^{e}Median length of plasma cell-free DNA fragments.

TABLE 5
Sequencing metrics for germline DNA
libraries analyzed by CAPP-Seq.

| | | | | | Median |
|---|---|---|---|---|---|
| | | | | Median | fragment |
| | NGS | On-target | Median | unique | length |
| Patient ID | reads^{a} | rate^{b} | depth^{c} | depth^{d} | (bp)^{e} |
| BC1045 | 80155852 | 52% | 10648 | 656 | 307 |
| BC1050 | 47732586 | 29% | 3761 | 491 | 275 |
| BC1058 | 88216385 | 52% | 11694 | 657 | 290 |
| BC1062 | 63855878 | 22% | 3728 | 1028 | 238 |
| BC1063 | 82548202 | 50% | 10587 | 621 | 258 |
| BC1064 | 45976357 | 28% | 3498 | 394 | 246 |
| BC1065 | 58252751 | 20% | 3213 | 880 | 256 |
| BC1069 | 53840485 | 27% | 3953 | 1064 | 247 |
| BC1071 | 41983397 | 31% | 3668 | 446 | 251 |
| BC1076 | 47472877 | 33% | 4309 | 535 | 271 |
| BC1077 | 80035043 | 49% | 10034 | 537 | 266 |
| BC1083 | 50816662 | 32% | 4372 | 531 | 261 |
| BC1085 | 53321221 | 29% | 4277 | 485 | 260 |
| BC1088 | 79618718 | 53% | 11918 | 1017 | 239 |
| BC1090 | 49898037 | 28% | 3790 | 432 | 242 |
| BC1096 | 54165553 | 27% | 3947 | 393 | 225 |
| BC1104 | 66777514 | 53% | 10097 | 964 | 235 |
| BC1105 | 74350224 | 50% | 9582 | 530 | 258 |
| BC1108 | 55329481 | 23% | 3494 | 389 | 239 |
| BC1109 | 54869415 | 27% | 3994 | 389 | 220 |
| BC1111 | 81563526 | 42% | 9332 | 779 | 230 |
| BC1116 | 52360249 | 27% | 3880 | 412 | 254 |
| BC1132 | 47569564 | 28% | 3589 | 379 | 250 |
| BC1133 | 96716658 | 44% | 11832 | 1084 | 253 |
| BC1135 | 113353007 | 55% | 15796 | 619 | 277 |
| BC1140 | 82737421 | 49% | 11494 | 965 | 247 |
| BC1147 | 63991557 | 51% | 9142 | 918 | 235 |
| BC1171 | 88816352 | 51% | 11254 | 668 | 273 |
| BC1174 | 83408547 | 61% | 14788 | 1293 | 233 |
| BC1182 | 87683304 | 52% | 11625 | 580 | 237 |
| BC1185 | 58405670 | 52% | 7588 | 307 | 222 |
| BC1186 | 102822647 | 51% | 13135 | 746 | 257 |
| BC1188 | 83633874 | 49% | 11425 | 970 | 252 |
| BC1196 | 46315525 | 50% | 6423 | 824 | 300 |
| BC1197 | 40067620 | 49% | 5295 | 614 | 253 |
| BC1198 | 43909892 | 51% | 6140 | 789 | 281 |
| BC1202 | 43618303 | 53% | 6307 | 793 | 262 |
| BC1203 | 48289424 | 38% | 5052 | 657 | 242 |
| BC1205 | 49776300 | 52% | 7073 | 991 | 260 |
| BC1206 | 47031765 | 50% | 6462 | 728 | 228 |
| BC1207 | 56854882 | 43% | 6667 | 896 | 260 |
| BC1209 | 66631578 | 42% | 7627 | 1207 | 268 |
| BC1215 | 48129509 | 64% | 8598 | 965 | 256 |
| BC1222 | 51264618 | 62% | 8623 | 1797 | 252 |
| BC1229 | 37043391 | 65% | 6611 | 710 | 249 |
| BC1233 | 56316730 | 62% | 9432 | 1787 | 250 |
| BC1235 | 46266078 | 66% | 8523 | 869 | 263 |
| BC1242 | 40795916 | 67% | 7543 | 743 | 248 |
| BC1243 | 40086334 | 66% | 7413 | 776 | 253 |
| BC1255 | 44169194 | 66% | 8031 | 867 | 255 |
| BC1256 | 39121496 | 66% | 7278 | 694 | 249 |
| BC1260 | 47704392 | 65% | 8696 | 874 | 264 |
| BC1265 | 49641830 | 63% | 7804 | 1396 | 238 |
| BC1269 | 47652797 | 65% | 8609 | 942 | 274 |
| BC1274 | 49324927 | 66% | 9095 | 949 | 263 |
| BC1275 | 43253853 | 65% | 7762 | 893 | 279 |
| BC1277 | 80481074 | 65% | 14616 | 1464 | 263 |
| BC1282 | 55695509 | 68% | 10231 | 2163 | 322 |
| BC1284 | 57270391 | 68% | 10465 | 2112 | 318 |
| BC1291 | 46784346 | 59% | 7439 | 1694 | 314 |
| BC1295 | 53844410 | 59% | 8571 | 1834 | 330 |
| BC1296 | 46107550 | 57% | 7125 | 1564 | 336 |
| BC1298 | 54109979 | 66% | 9420 | 1812 | 344 |
| BC1300 | 47794193 | 66% | 8400 | 1706 | 343 |
| BC1303 | 53173654 | 59% | 8453 | 1479 | 258 |
| BC1304 | 53853002 | 59% | 8555 | 1797 | 316 |
| BC1308 | 53957106 | 63% | 8631 | 1345 | 255 |
| BC1309 | 51580182 | 63% | 8233 | 1400 | 242 |
| BC1310 | 57371748 | 62% | 9022 | 1616 | 253 |
| BC1312 | 61732421 | 61% | 9602 | 1380 | 255 |
| BC1313 | 50959433 | 61% | 7841 | 1296 | 248 |
| BC1314 | 62280751 | 60% | 9314 | 1327 | 265 |
| BC1315 | 53887467 | 60% | 8198 | 1351 | 250 |
| BC1316 | 52269840 | 60% | 8106 | 1224 | 250 |

^{a}Number of total sequencing reads per sample after demultiplexing.
^{b}Percentage of quality-control-passed reads overlapping with the targeted hybrid-capture panel.
^{c}Median non-deduplicated sequencing depth.
^{d}Median UMI-deduplicated sequencing depth.
^{e}Median length of sonicated DNA fragments.

ULP-WGS libraries prepared from urine cfDNA were sequenced to a median unique coverage of 2× (see e.g., TABLE 6).

TABLE 6
Sequencing metrics and inferred tumor
fractions for ULP-WGS libraries.

| | | Median | CNA-inferred |
|---|---|---|---|
| | Deduplicated | deduplicated | tumor fraction |
| Patient ID | NGS reads^{a} | depth^{b} | (%)^{c} |
| BC1045 | 16192495 | 2 | 2.1 |
| BC1050 | 23300535 | 2 | 2.5 |
| BC1058 | 26498913 | 2 | 4.1 |
| BC1062 | 32777749 | 2 | 1.6 |
| BC1063 | 39793732 | 3 | 3.9 |
| BC1064 | 26109470 | 2 | 0.0 |
| BC1065 | 15265673 | 2 | 1.0 |
| BC1069 | 35059531 | 3 | 12.9 |
| BC1071 | 31413690 | 2 | 0.0 |
| BC1076 | 7797202 | 3 | 6.8 |
| BC1077 | 23385225 | 2 | 13.9 |
| BC1083 | 23023301 | 3 | 7.8 |
| BC1085 | 25676345 | 2 | 49.3 |
| BC1088 | 47240517 | 3 | 9.1 |
| BC1090 | 22915385 | 2 | 14.6 |
| BC1096 | 31848372 | 3 | 50.0 |
| BC1104 | 21721060 | 2 | 2.2 |
| BC1105 | 16686498 | 2 | 0.0 |
| BC1108 | 24846207 | 2 | 0.0 |
| BC1109 | 41532938 | 3 | 0.0 |
| BC1111 | 43739787 | 3 | 4.8 |
| BC1116 | 30812303 | 3 | 24.4 |
| BC1132 | 13674139 | 2 | 1.4 |
| BC1133 | 23536702 | 2 | 24.0 |
| BC1135 | 19263791 | 2 | 9.9 |
| BC1140 | 24670655 | 2 | 22.7 |
| BC1147 | 40747026 | 3 | 3.6 |
| BC1171 | 14700321 | 2 | 45.9 |
| BC1174 | 21241187 | 2 | 8.0 |
| BC1182 | 20361029 | 2 | 19.4 |
| BC1185 | 15055460 | 2 | 2.4 |
| BC1186 | 12164397 | 2 | 13.8 |
| BC1188 | 27737907 | 3 | 16.0 |
| BC1196 | 26916870 | 3 | 5.9 |
| BC1197 | 24746508 | 2 | 1.8 |
| BC1198 | 18739602 | 2 | 0.0 |
| BC1202 | 16377651 | 2 | 1.6 |
| BC1203 | 24686012 | 2 | 0.0 |
| BC1205 | 26406299 | 2 | 4.1 |
| BC1206 | 20231127 | 4 | 5.8 |
| BC1207 | 15944674 | 2 | 0.0 |
| BC1209 | 27422601 | 2 | 8.4 |
| BC1215 | 17478058 | 2 | 0.0 |
| BC1222 | 6186128 | 2 | 9.8 |
| BC1229 | 14109229 | 2 | 9.4 |
| BC1233 | 21501231 | 2 | 61.6 |
| BC1235 | 20460671 | 2 | 2.5 |
| BC1242 | 17895486 | 2 | 2.8 |
| BC1243 | 21417820 | 2 | 0.0 |
| BC1255 | 21155013 | 2 | 58.3 |
| BC1256 | 15398406 | 2 | 0.0 |
| BC1260 | 22326975 | 2 | 21.0 |
| BC1265 | 15337874 | 2 | 6.1 |
| BC1269 | 20894234 | 2 | 4.8 |
| BC1274 | 13244367 | 2 | 6.0 |
| BC1275 | 11743124 | 2 | 17.1 |
| BC1277 | 13964173 | 2 | 0.0 |
| BC1282 | 12486959 | 2 | 4.5 |
| BC1284 | 19318703 | 2 | 2.8 |
| BC1291 | 28713660 | 2 | 12.9 |
| BC1295 | 15650859 | 2 | 3.7 |
| BC1296 | 32133412 | 2 | 0.0 |
| BC1298 | 52135118 | 4 | 3.4 |
| BC1300 | 22132074 | 2 | 0.0 |
| BC1303 | 19276831 | 2 | 1.3 |
| BC1304 | 26273724 | 2 | 14.0 |
| BC1308 | 18237120 | 2 | 55.9 |
| BC1309 | 17185809 | 2 | 0.0 |
| BC1310 | 18952011 | 2 | 11.6 |
| BC1312 | 24500473 | 2 | 0.0 |
| BC1313 | 28850682 | 3 | 12.9 |
| BC1314 | 25024775 | 2 | 0.0 |
| BC1315 | 26415324 | 2 | 20.9 |
| BC1316 | 21176619 | 2 | 3.9 |

^{a}Samtools-deduplicated properly paired reads.
^{b}Median deduplicated sequencing depth across the whole genome.
^{c}Tumor fraction estimated from urine cell-free DNA WGS copy number alterations using ichorCNA.

Cell-Free DNA Biomarker Differences in Relation to pCR Status

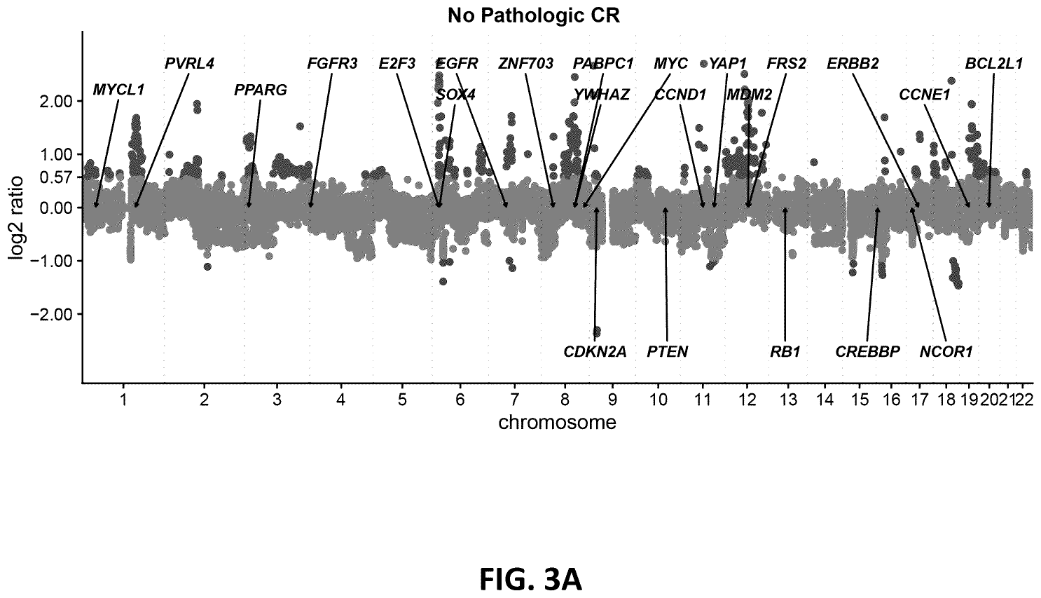

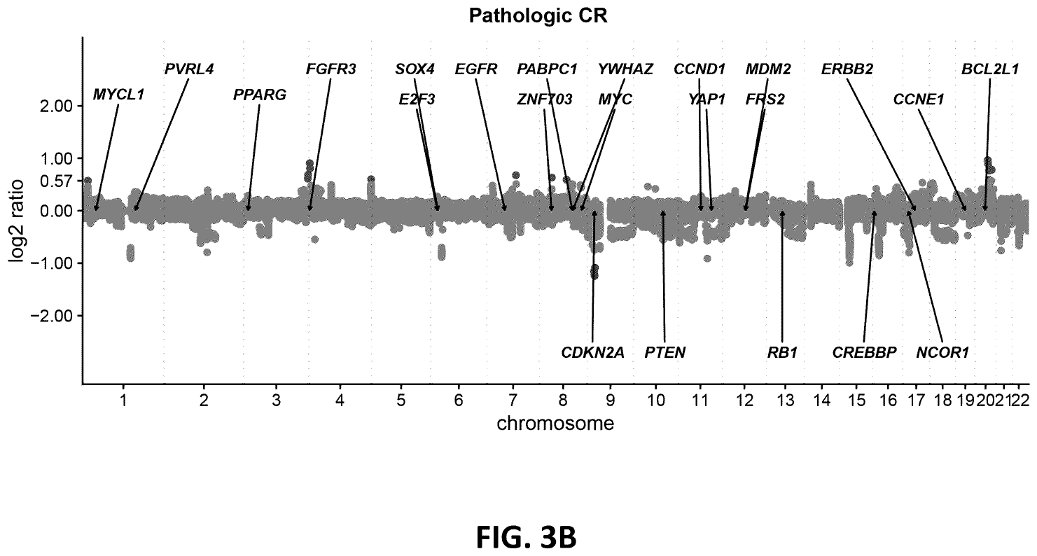

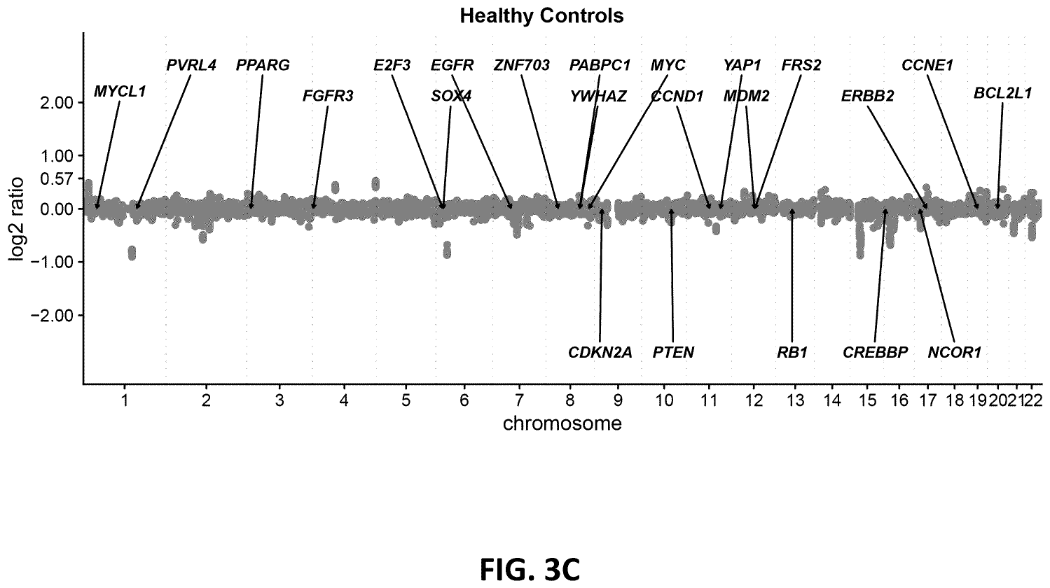

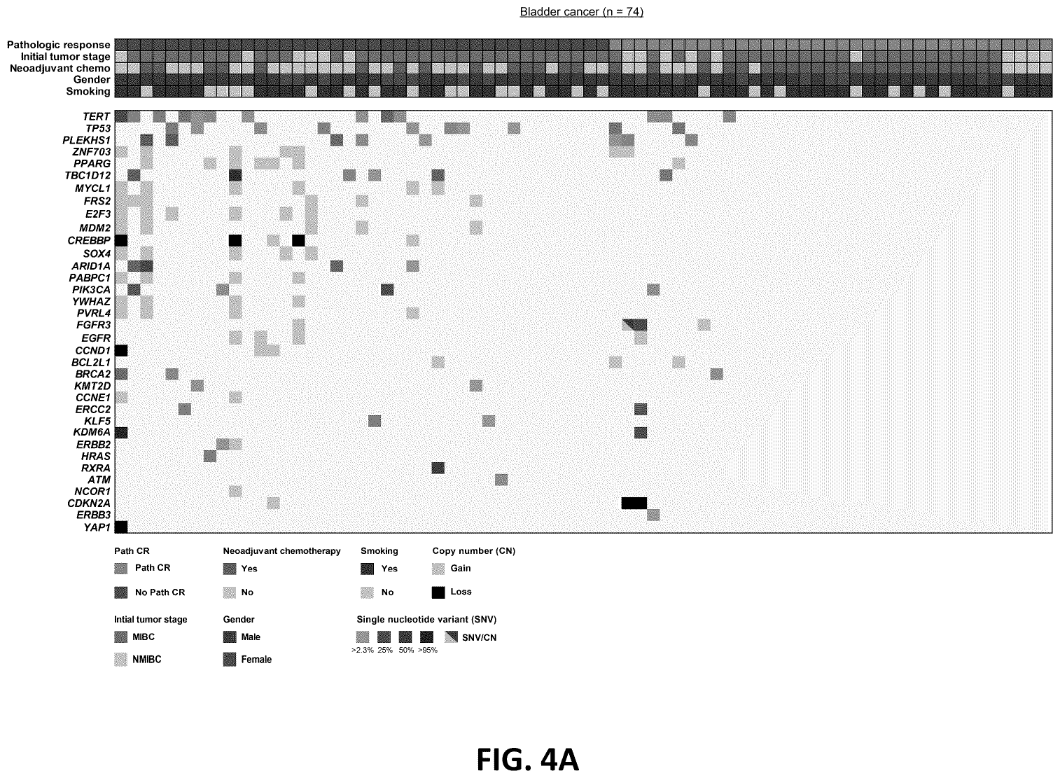

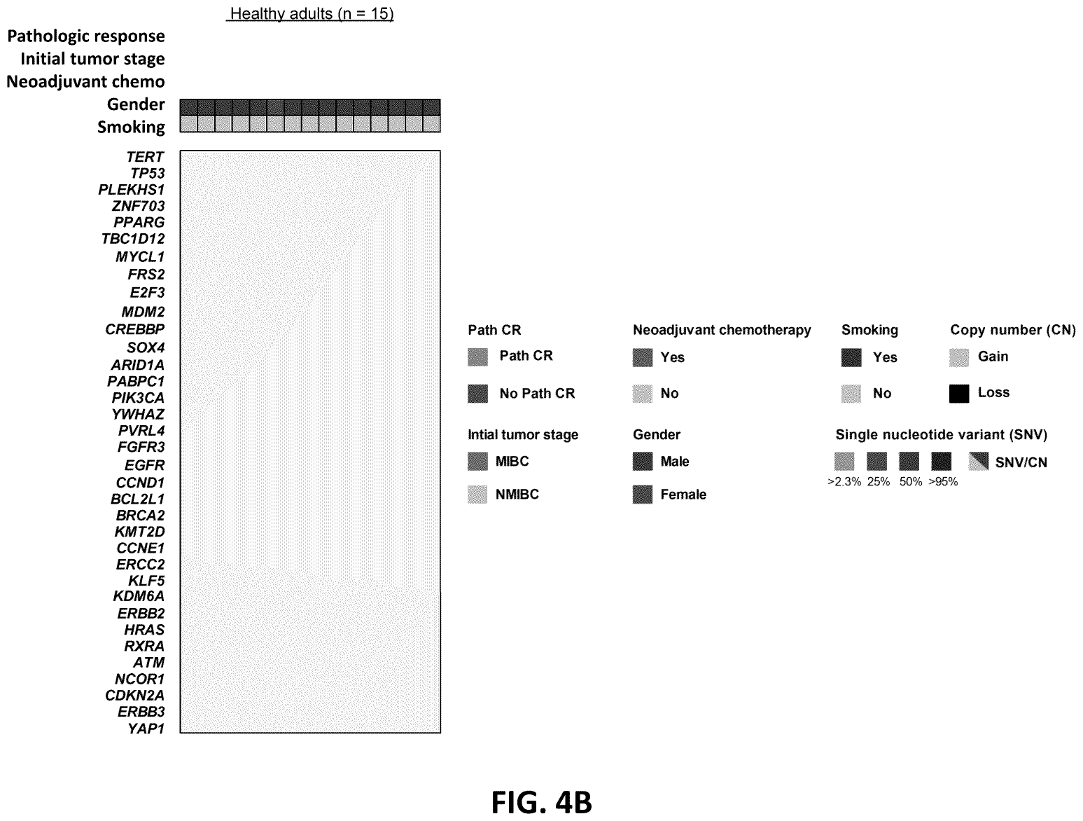

Copy number-derived tumor fraction (TFx) levels, estimated from ULP-WGS of urine cfDNA, ranged from 0 to 62% with a median value of 4.3% in this cohort (see e.g., TABLE 2). Genome-wide analysis of urine cfDNA revealed focal copy number alteration of genes previously reported by The Cancer Genome Atlas (TCGA) to be recurrently altered in MIBC (see e.g., FIG. 3A-FIG. 3C) with PPARG, ZNF703, and E2F3 being the most frequently amplified. Further, uCAPP-Seq analysis of single nucleotide variant (SNV) data from the full 74 patient cohort revealed that the TERT promotor and TP53 were the most commonly mutated genes (see e.g., FIG. 4A-FIG. 4B), again consistent with prior tissue sequencing data. Indicative of specificity, neither copy number alterations nor SNVs were detected with significance in healthy adult urine cfDNA (see e.g., FIG. 3A-FIG. 3C and FIG. 4A-FIG. 4B). Additionally, results of the copy number (see e.g., FIG. 3A-FIG. 3C) and uCAPP-Seq (see e.g., FIG. 4A-FIG. 4B) analyses demonstrated clear differences in urine cfDNA based on pathologic complete response (pCR) status, which was determined by examination of surgical specimens by board-certified genitourinary pathologists.

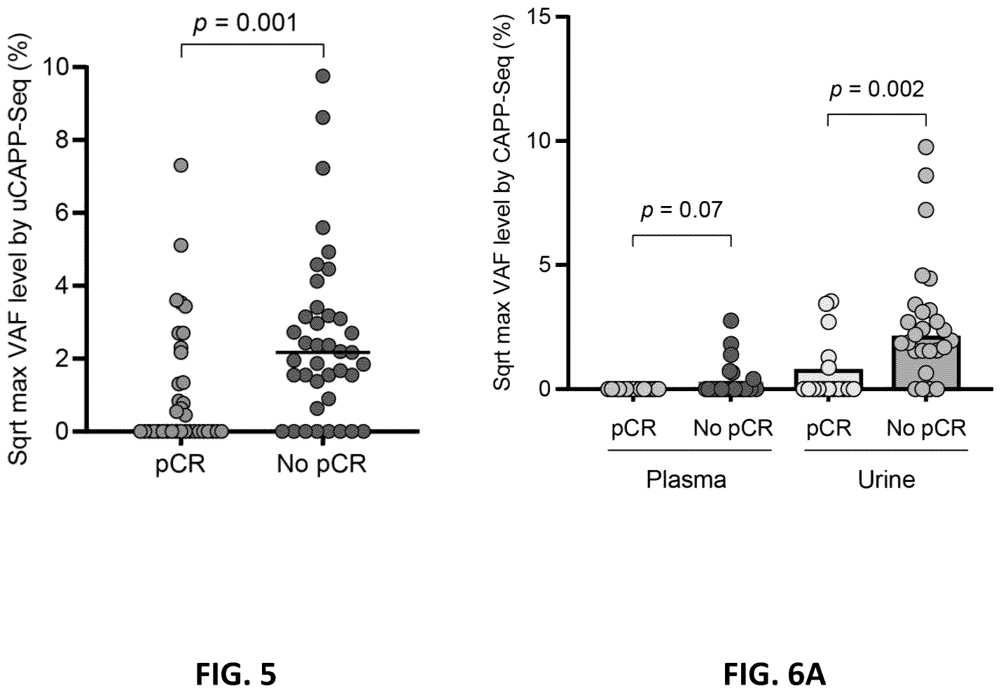

Bladder cancer patients who achieved pCR had significantly lower variant allele frequency (VAF) levels measured by uCAPP-Seq compared to those who did not (see e.g., FIG. 5) despite having similar baseline characteristics (see e.g., TABLE 7).

TABLE 7
Clinical characteristics according to pathologic response status.

| | Patient characteristics (n = 74) | | |
|---|---|---|---|
| | pCR | No pCR | |
| | (n = 35) | (n = 39) | P value |
| Gender | | | |
| Male | 27 | 31 | 1.00 |
| Female | 8 | 8 | |
| Median age | 65 | 69 | 0.78 |
| (years) | | | |
| Ethnicity | | | |
| White | 34 | 34 | 0.20 |
| Non-white | 1 | 5 | |
| Smoking history | | | |
| yes | 28 | 23 | 0.08 |
| no | 7 | 16 | |
| T-stage (pre-treatment)^{a} | | | |
| Ta | 1 | 1 | |
| Tis | 2 | 0 | |
| T1 | 8 | 4 | 0.09 |
| T2 | 22 | 30 | |
| T3 | 2 | 4 | |
| Neoadjuvant chemotherapy received | | | |
| yes | 23 | 15 | |
| ddMVAC | 6 | 3 | |
| gemcitabine/cisplatin | 12 | 9 | |
| gemcitabine/carboplatin | 1 | 0 | |
| cisplatin/etoposide | 1 | 0 | 0.02 |
| carboplatin/paclitaxel | 1 | 0 | |
| treatment change | 1 | 0 | |
| unknown regimen | 1 | 3 | |
| no | 12 | 24 | |
| Histology | | | |
| urothelial | 34 | 34 | |
| other | 1 | 5 | 0.20 |

^{a}T staging was performed at the time of pre-treatment TURBT using AJCC 8 criteria.

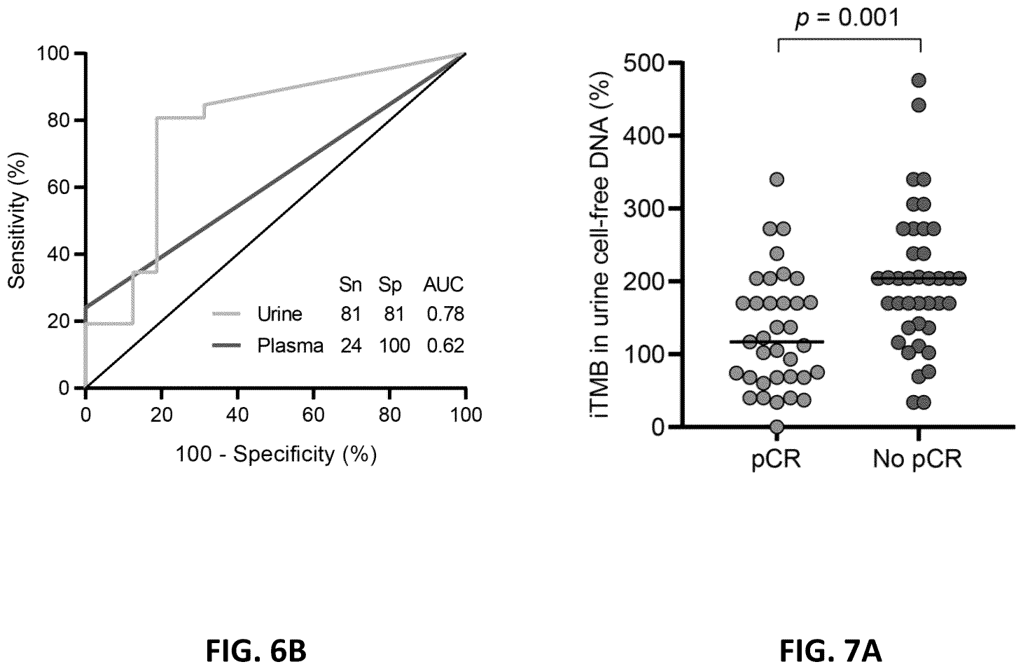

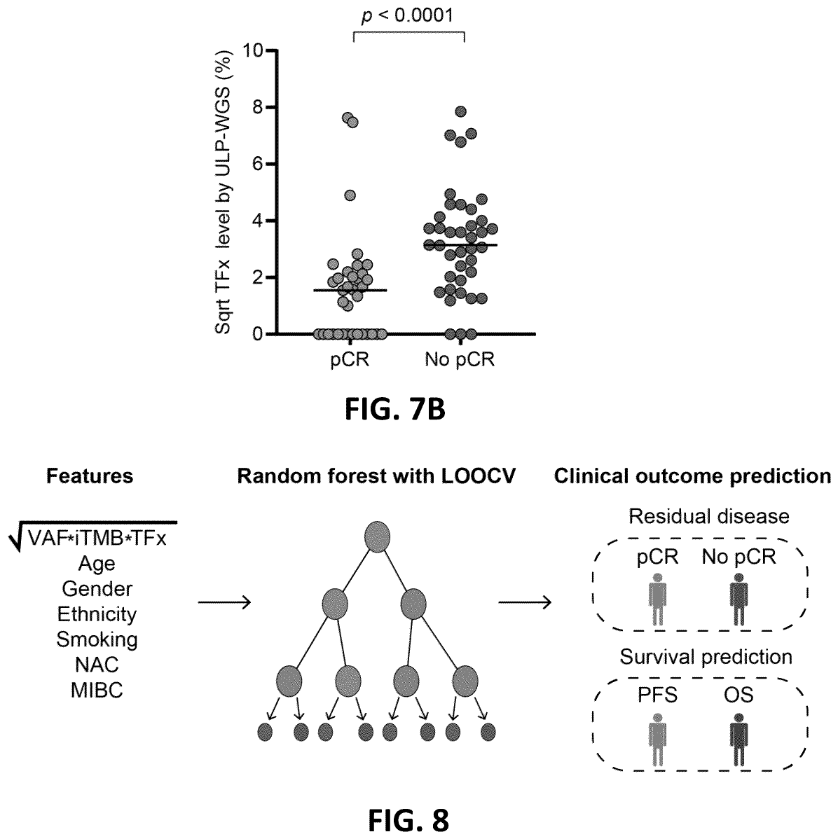

Strikingly, urine cfDNA significantly outperformed plasma circulating tumor DNA (see e.g., FIG. 6A-FIG. 6B). The tumor mutational burden inferred from the number of non-silent mutations detected in urine cfDNA (iTMB) was also measured. The median iTMB was 170 (range 0-476) across the cohort, consistent with previous reports in bladder cancer. Comparing between subgroups, patients with no pCR had significantly higher iTMB levels than patients with pCR (median 204 vs. 117, p=0.001) (see e.g., FIG. 7A). This result is consistent with findings in breast cancer, suggesting that increased TMB is a negative predictor of pCR to neoadjuvant chemotherapy. TFx, which was inferred from genome-wide copy number alterations in urine cfDNA, also differed significantly based on pCR status (median 2.4% for pCR vs. 9.9% for no pCR, p<0.0001) (see e.g., FIG. 7B) suggesting that genome-wide copy number alterations, like SNVs, could be utilized for urine-based MRD detection in bladder cancer.

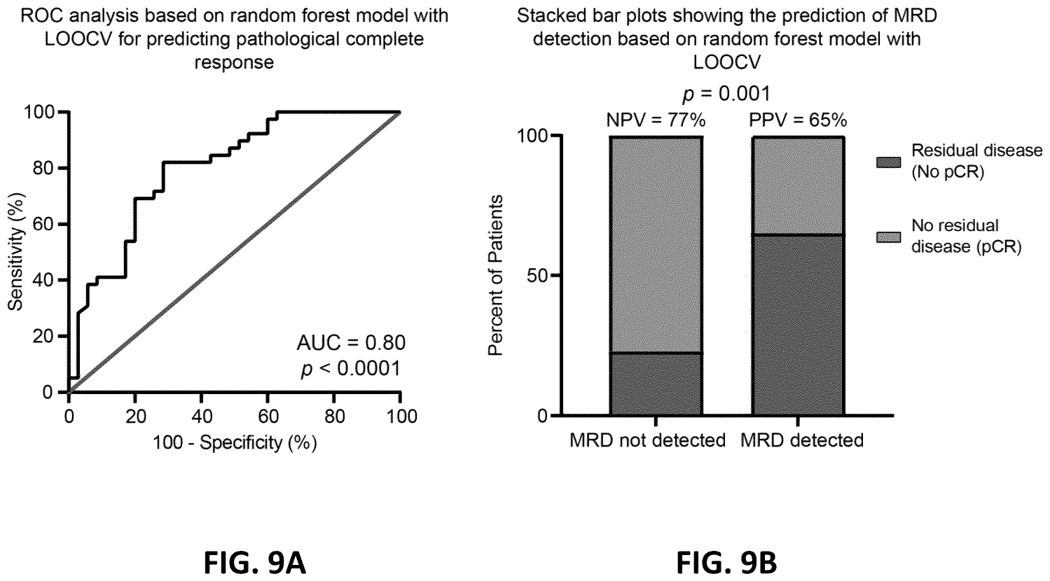

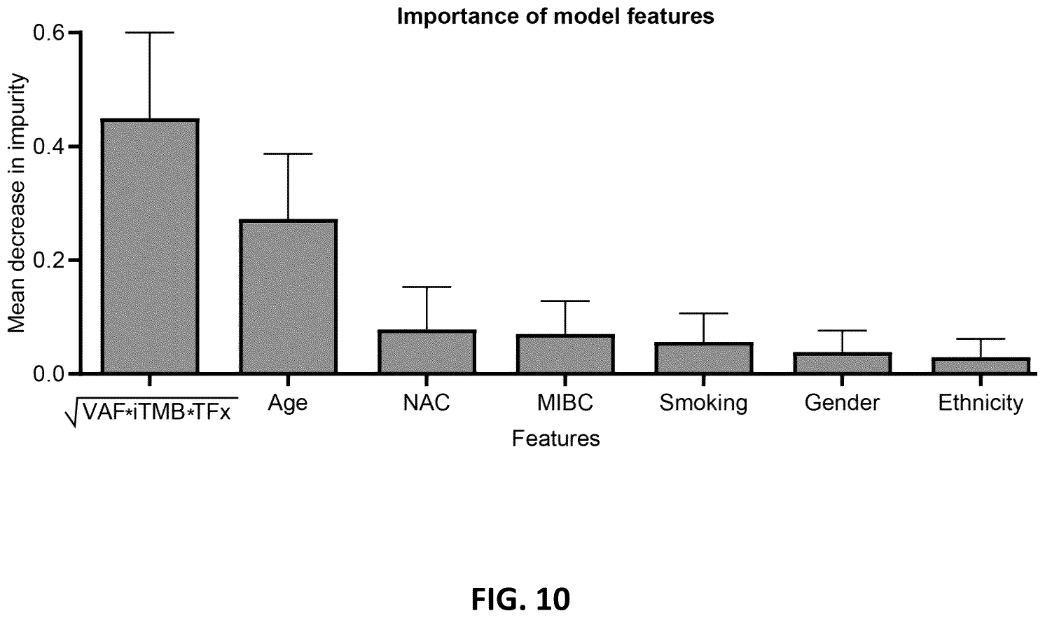

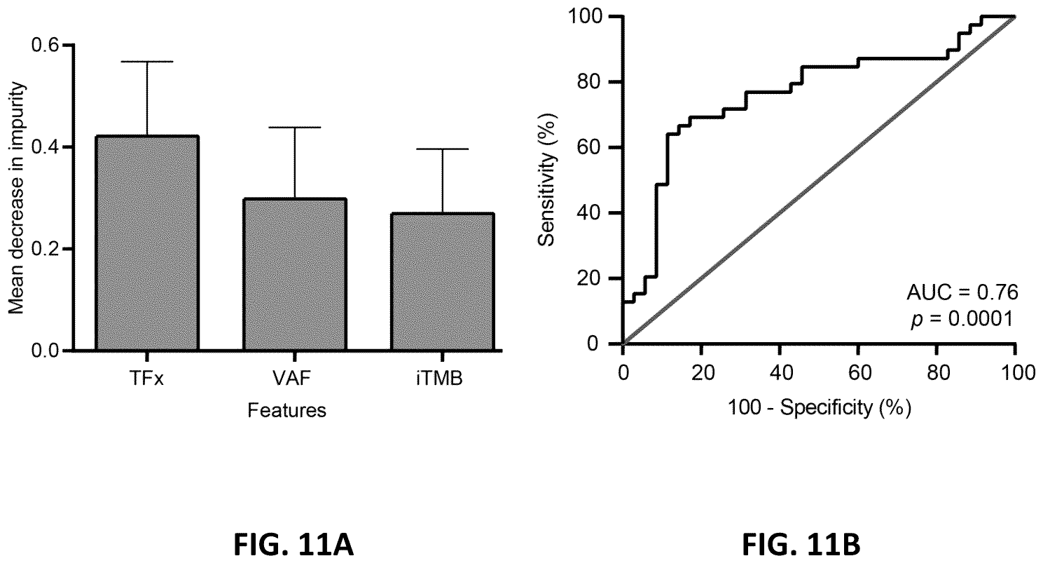

Random forest model for pCR and survival prediction Next the three urine cfDNA-derived metrics—maximum VAF, iTMB, and TFx—were integrated with pretreatment clinical variables using a machine learning random forest model that was validated by leave-one-out cross-validation (LOOCV) (see e.g., FIG. 8). Area under the receiver operating characteristic curve (AUROC) for the random forest model was 0.80 (p<0.0001) (see e.g., FIG. 9A), with a sensitivity of 87%, a negative predictive value (NPV) of 77%, and a positive predictive value (PPV) of 65% for determining pCR (see e.g., FIG. 9B). The combinatorial urine cfDNA metric was by far the most important predictive feature in the model (see e.g., FIG. 10). Indeed, when a LOOCV model was developed including only urine cfDNA features (maximum VAF, iTMB, and TFx), its performance remained high with AUROC of 0.76 for determining pCR (see e.g., FIG. 11A-FIG. 11B).

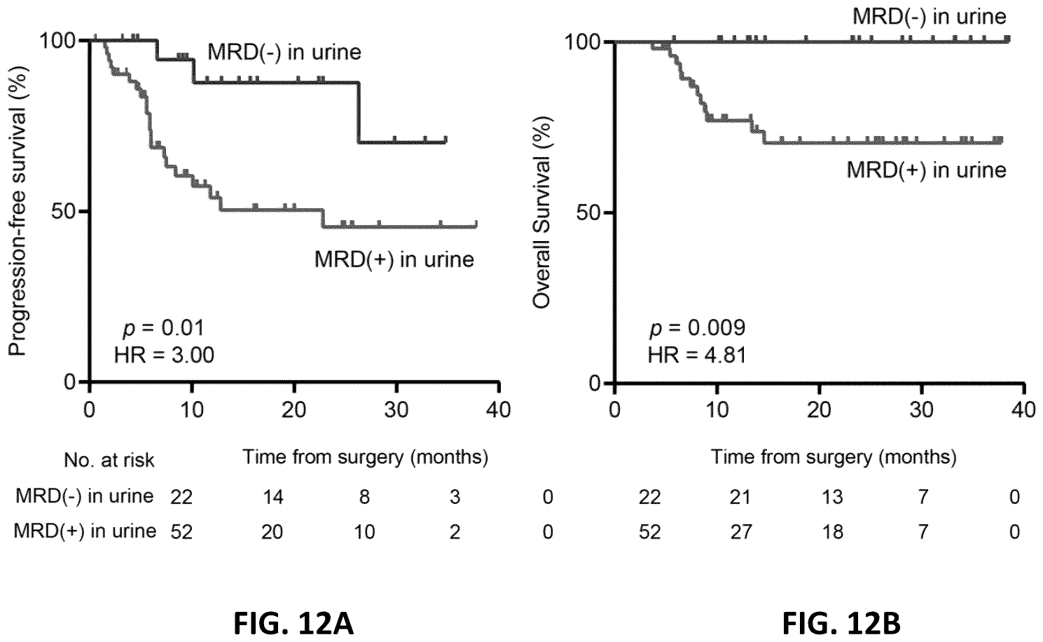

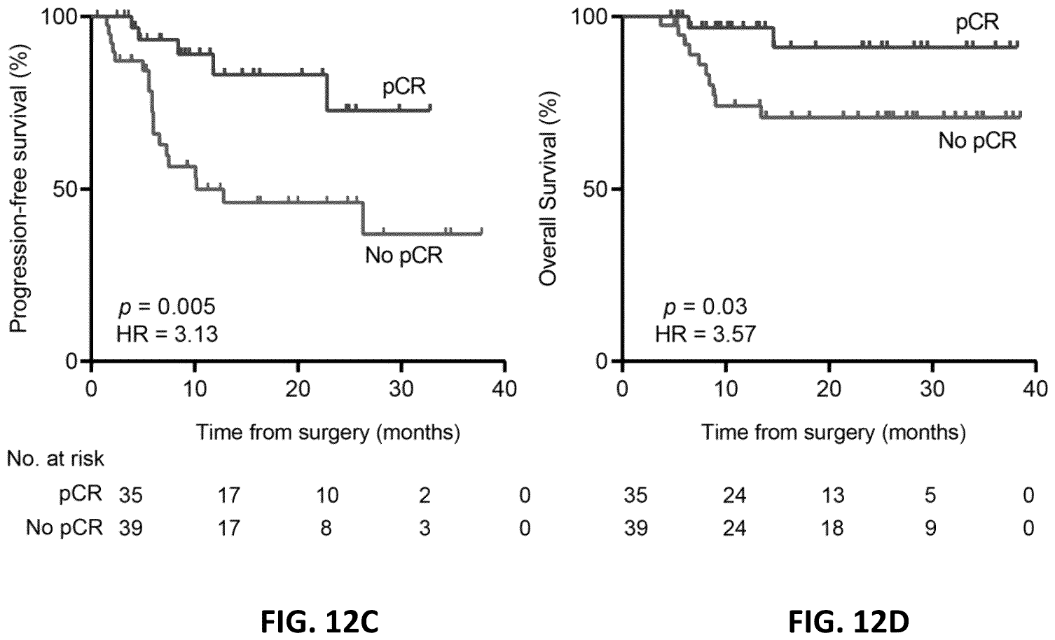

Using the LOOCV model, it was aimed to predict survival outcomes within the 74-patient localized bladder cancer cohort. Therefore, Kaplan-Meier and Cox regression landmark analyses were performed starting from the time of surgery (see e.g., FIG. 12A-FIG. 12D, TABLE 8, and TABLE 9).

TABLE 8
Univariate Cox regressions for progression-free and overall survival.

| | Progression-free survival | | | Overall survival | | |
|---|---|---|---|---|---|---|
| Variable | HR | 95% CI | P value | HR | 95% CI | P value |
| Age | 0.96 | 0.92-1.01 | 0.08 | 0.97 | 0.92-1.03 | 0.37 |
| Gender | 2.27 | 0.07-7.63 | 0.19 | 1.43 | 0.31-6.53 | 0.65 |
| (male/female) | | | | | | |
| Ethnicity | 1.45 | 0.49-4.32 | 0.51 | 1.84 | 0.49-6.91 | 0.37 |
| (white/non- | | | | | | |
| white) | | | | | | |
| Neoadjuvant | 0.95 | 0.43-2.12 | 0.91 | 0.46 | 0.14-1.52 | 0.20 |
| chemotherapy | | | | | | |
| (yes/no) | | | | | | |
| MIBC | 2.05 | 0.61-6.90 | 0.25 | * | * | * |
| (yes/no) | | | | | | |
| Smoking | 0.32 | 0.14-0.72 | 0.01 | 0.28 | 0.09-0.87 | 0.03 |
| (yes/no) | | | | | | |
| VAF (uCAPP- | 1.01 | 0.99-1.03 | 0.51 | 1.02 | 1.00-1.04 | 0.10 |
| Seq)ª | | | | | | |
| ITMB | 1.00 | 0.99-1.01 | 0.16 | 1.01 | 1.00-1.01 | 0.06 |
| (uCAPP- | | | | | | |
| Seq)^{b} | | | | | | |
| TFX (ULP- | 1.02 | 1.00-1.05 | 0.05 | 1.03 | 1.00-1.06 | 0.03 |
| WGS)^{c} | | | | | | |
| RF model | 4.35 | 1.29-14.70 | 0.02 | * | * | * |
| (LOOCV)^{d} | | | | | | |

^{a}SNV-derived maximum non-silent variant allele frequency (VAF) in urine cell-free DNA, determined by uCAPP-Seq.
^{b}Exome-wide inferred tumor mutational burden interpolated from uCAPP-Seq results.
^{c}Copy number-inferred tumor fraction (TFx) estimated from ULP-WGS of urine cell-free DNA.
^{d}Random forest (RF) model with leave-one-out cross-validation (LOOCV) as described in FIG. 8.
RF model was coded as a continuous variable based on the predicted probability of residual disease for each case (0-1.0).
RF model HR represents risk of event associated with 0.01 increase in probability of residual disease.
*No death events observed in individuals predicted to achieve pCR by the LOOCV RF model, or in those with non-MIBC.

TABLE 9
Multivariate Cox regression for progression-free and overall
survival.

| | Progression-free survival | | | Overall survival | | |
|---|---|---|---|---|---|---|
| | | | p | | | p |
| Variable | HR | 95% CI | value | HR | 95% CI | value |
| RF model | 4.26 | 1.25-14.52 | 0.02 | * | * | * |
| (LOOCV)ª | | | | | | |
| Hematocrit | 0.99 | 0.93-1.06 | 0.87 | 0.97 | 0.88-1.06 | 0.54 |
| (%) | | | | | | |
| Body mass | 0.93 | 0.85-1.02 | 0.13 | 0.88 | 0.77-1.01 | 0.07 |
| index (kg/m²) | | | | | | |
| Urine cfDNA | 1.00 | 0.99-1.00 | 0.79 | 1.00 | 0.99-1.00 | 0.90 |
| (ng/ml) | | | | | | |

^{a}Random forest (RF) model with leave-one-out cross-validation (LOOCV) as described in FIG. S5A.
RF model was coded as a continuous variable based on the predicted probability of residual disease for each case (0-1.0).
RF model HR represents risk of event associated with 0.01 increase in probability of residual disease.
*No death events observed in individuals predicted to achieve pCR by the LOOCV RF model.

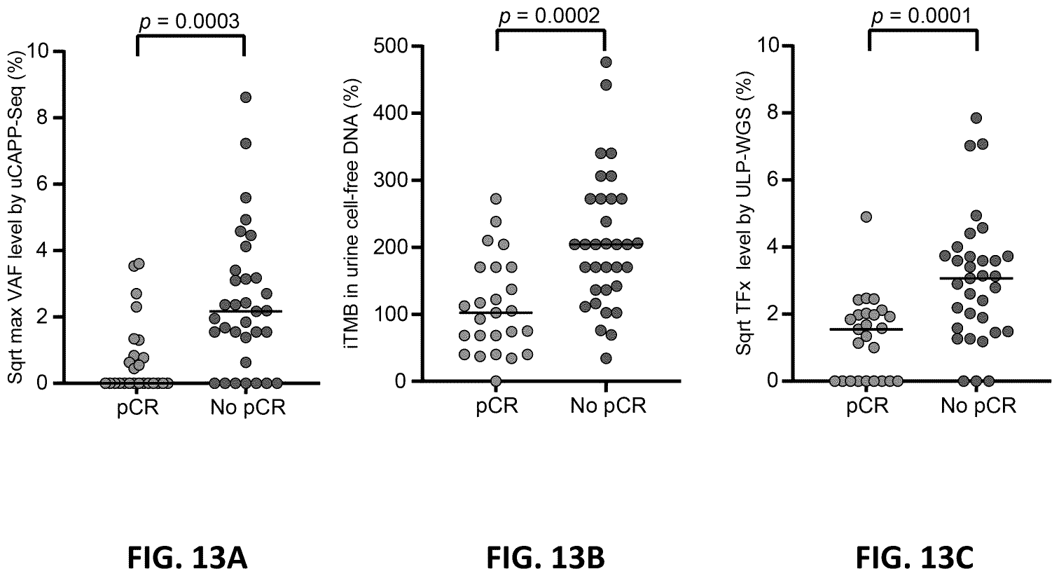

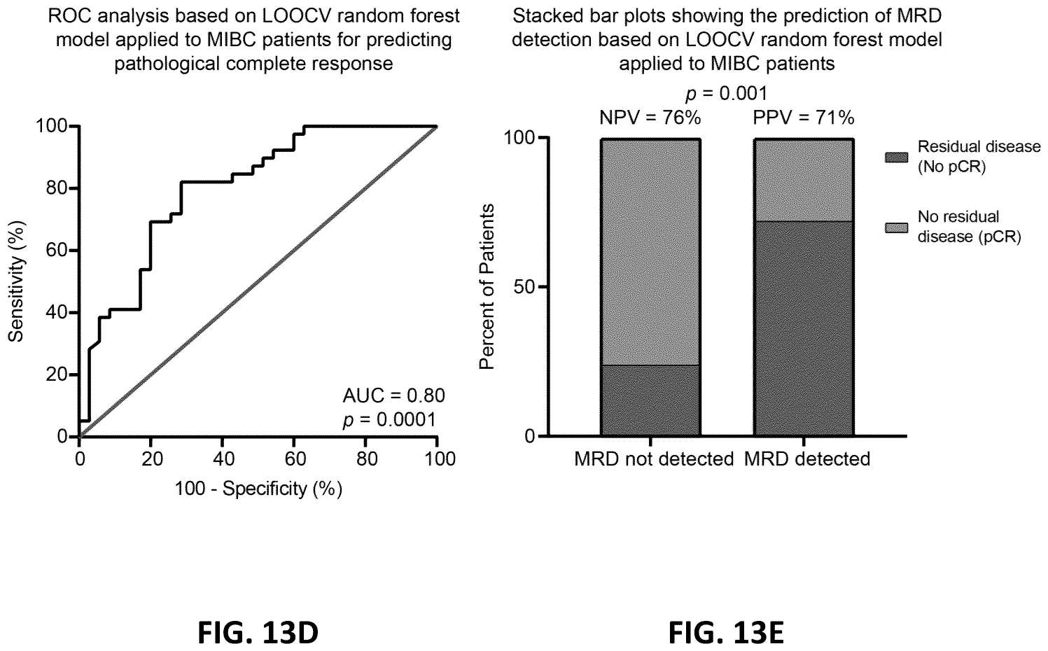

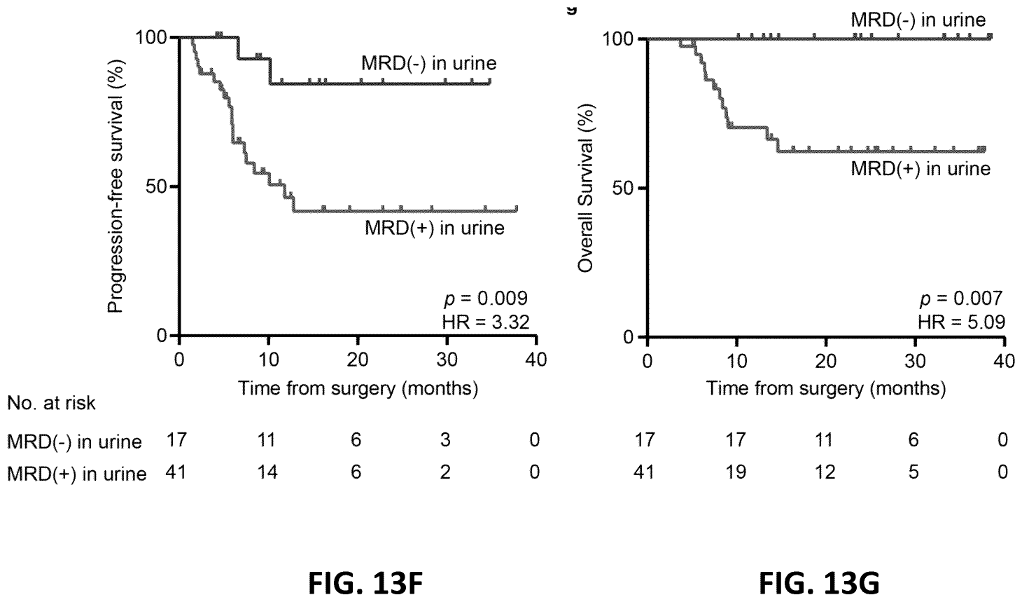

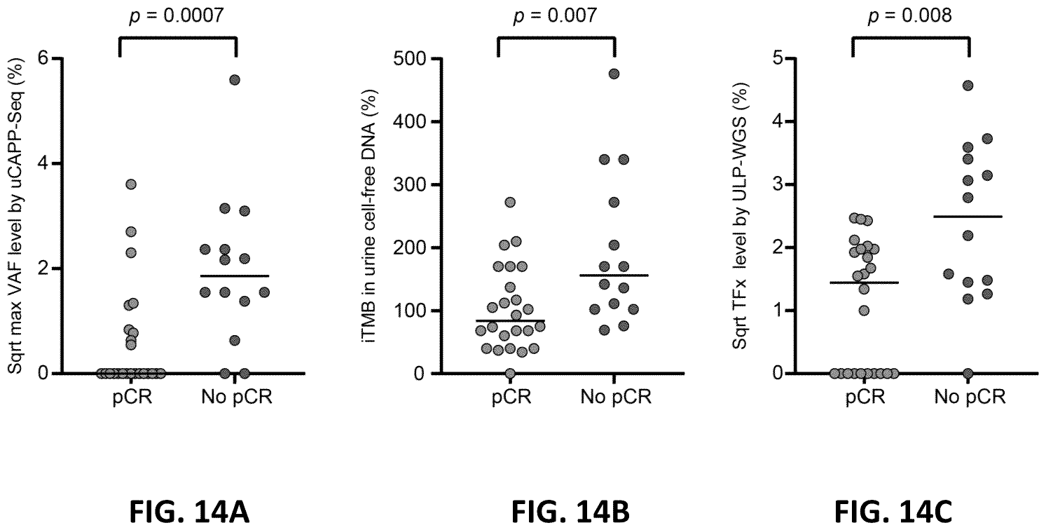

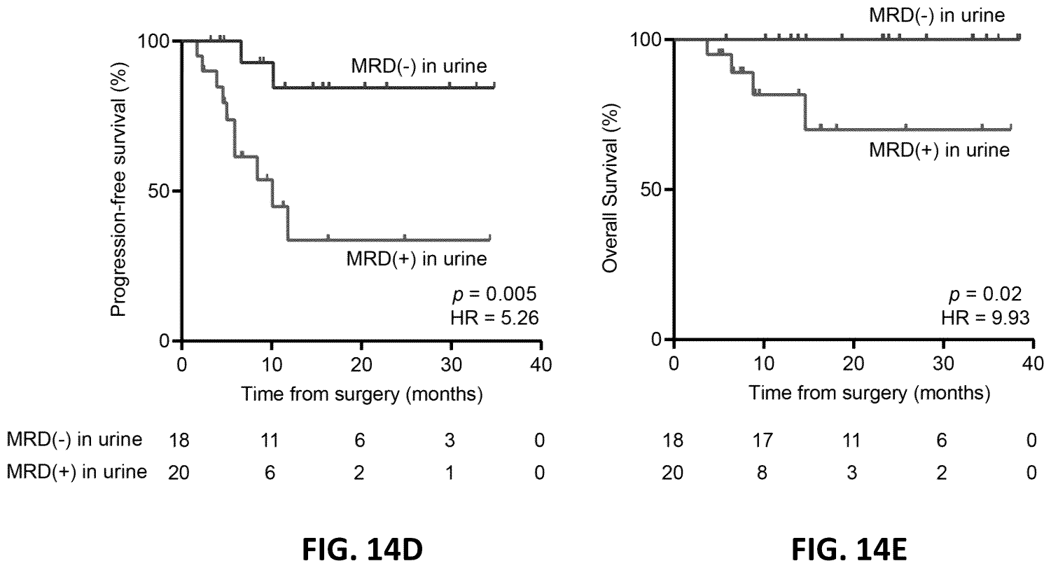

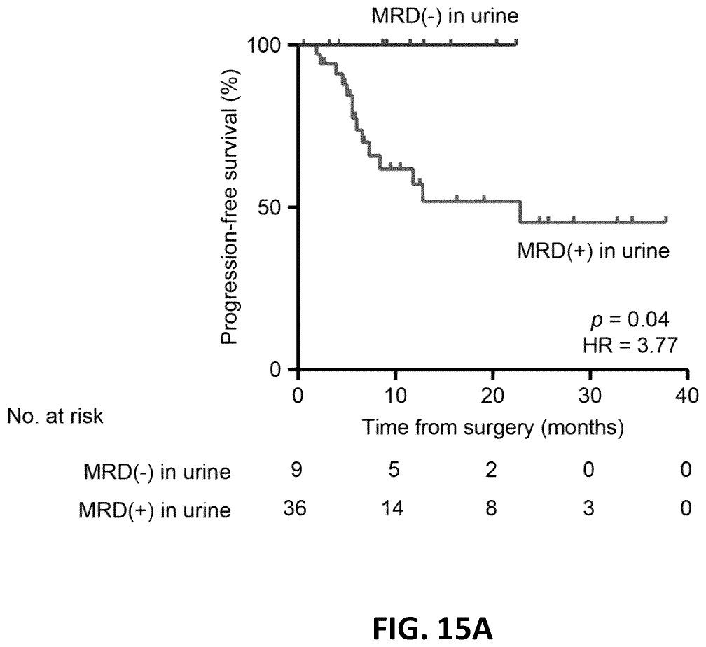

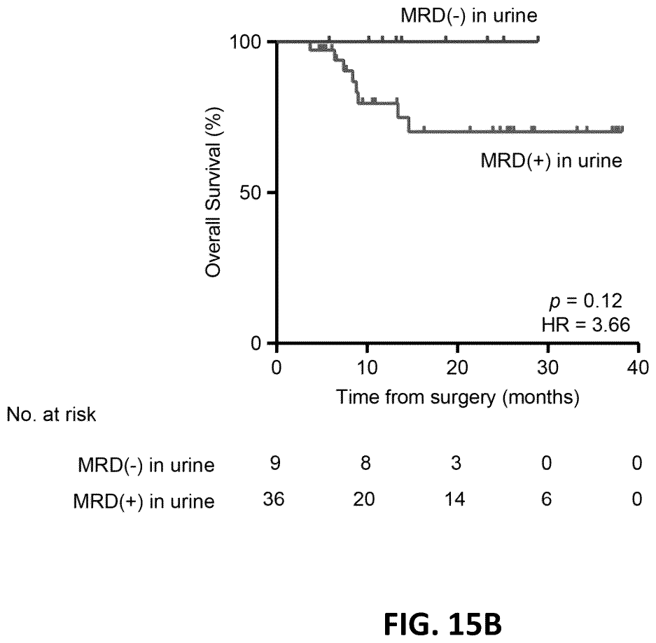

Strikingly, patients predicted by the model to harbor MRD also had significantly worse progression-free survival (PFS) (HR=3.00, p=0.01; see e.g., FIG. 12A) and overall survival (OS) (HR=4.81, p=0.009; see e.g., FIG. 12B), comparable to the presence of residual disease in the radical cystectomy specimen itself (PFS HR=3.13, p=0.005; OS HR=3.57, p=0.03; see e.g., FIG. 12C-FIG. 12D). Univariate and multivariate Cox proportional hazards models confirmed the significance of the MRD predictions (see e.g., TABLE 8 and TABLE 9). The model remained predictive for both PFS and OS when restricted to only MIBC patients (see e.g., FIG. 13A-FIG. 13G) and patients treated with NAC (see e.g., FIG. 14A-FIG. 14E). Furthermore, the model remained significant for predicting PFS when applied to an independent held-out validation cohort (see e.g., FIG. 15A) with a trend toward predicting OS significantly as well (see e.g., FIG. 15B).

Discussion

Herein was developed a multi-modal urine cfDNA method to sensitively detect MRD and predict pCR in bladder cancer patients. The technology also predicted survival significantly and comparably to gold-standard surgical pathologic analysis of resected tumor tissue. This study included patients having a single timepoint assessment of urine cfDNA. Other investigations utilizing plasma have shown that multiple samples obtained in surveillance settings may achieve greater sensitivity for detecting circulating tumor DNA MRD. Nevertheless, high MRD sensitivity was achieved by multimodally analyzing urine, the biofluid most proximal to localized bladder cancer. While the study was prospective, all samples were obtained from a single medical center. The findings may be further corroborated in a multi-institutional setting. Finally, given the prospective nature of the study with all patients enrolled between 2019 and 2021, the median follow-up time was 23 months. In future studies, a longer follow-up may be used to further confirm the dramatic survival differences observed.

In conclusion, the multi-omic urine-based cell-free DNA analysis allowed for the detection of MRD with high sensitivity and risk-stratified patients by survival. In the future, this type of integrative analysis could potentially be used to facilitate more personalized clinical decision-making for bladder cancer.

Methods

Patient Recruitment and Sample Collection

74 patients with localized bladder cancer who proceeded with curative-intent radical cystectomy at the Washington University Siteman Cancer Center were enrolled. Eligible patients were required to be at least 18 years old and to have a diagnosis of bladder cancer confirmed by histologic or cytologic assessment. Urine and blood collection was performed at the time of enrollment. Urine and blood samples from 15 healthy adult volunteers were also utilized for comparison. The methods were performed in accordance with relevant guidelines and regulations and approved by the institutional review board at the Washington University in St. Louis School of Medicine. Patients and healthy donors were enrolled in NCT04354064. Written informed consent was obtained from all trial participants in accordance with the Declaration of Helsinki. This study followed the Strengthening the Reporting of Observational Studies in Epidemiology (STROBE) guidelines for observational studies.

Pathologic Response Assessment

Surgical resection specimens from radical cystectomy procedures were processed consistently using a standardized institutional approach, including specimen collection, handling, and submission to the Pathology Department at the Washington University School of Medicine. Resected surgical specimens were microscopically reviewed by blinded board-certified genitourinary surgical pathologists. AJCC 8th edition pathologic stage TO, Tis, and Ta were defined as pathologic complete response (pCR) in the study. Non-pathologic complete response (no pCR) was defined as stages T1, T2, T3, or T4, with or without evidence of nodal disease (N1-N2) and/or evidence of metastatic disease.

Urine Cell-Free DNA Extraction

Urine samples were collected in cups pre-filled with 1-2 mL of 0.5M EDTA. Shortly following collection, cfDNA was extracted from 22 to 90 ml of urine with Q-sepharose resin slurry (GE Healthcare, Chicago, Illinois). Briefly, Q-sepharose resin was added to urine at a ratio of 10 ul slurry per ml of urine and mixed for 30 min. After centrifuging the mixture at 1800× g for 10 min, the supernatant was discarded. The resin was washed twice with 0.3M LiCl/10 mM sodium acetate (pH 5.5), transferred to a Micro Bio-Spin column (Bio-Rad, Hercules, California, USA), and the bound DNA was eluted with 70% ethanol and passed over a QIAquick column (Qiagen, Hilden, Germany). Columns were then washed with 2M LiC; in 70% ethanol, followed by 75 mM potassium acetate (pH 5.5) in 80% ethanol. Finally, DNA was eluted in nuclease-free water or 10 mM Tris-CI (pH 8.5). Urine cfDNA was quantified using the Qubit dsDNA High Sensitivity Assay kit (Thermo Fisher Scientific, Waltham, Massachusetts). cfDNA quality was assessed on an Agilent 2100 Bioanalyzer (Agilent Technologies, Santa Clara, California).

Germline DNA Extraction

A peripheral blood sample was collected from each subject using EDTA tubes (Becton Dickinson, Franklin Lakes, New Jersey). Plasma-depleted whole blood (PDWB) was collected by centrifugation and then frozen at −80° C. prior to the isolation of germline DNA. Germline DNA was extracted from 50 to 100 ul of PDWB using the QIAmp DNA Micro Kit (Qiagen, Hilden, Germany) according to the manufacturer's instructions. DNA was then quantified by the Qubit dsDNA High Sensitivity Assay to determine yield (Thermo Fischer, Waltham, Massachusetts).

Cancer Personalized Profiling by Deep Sequencing (CAPP-Seq)

Urine CAPP-Seq was performed on urine cfDNA along with matched germline DNA. Briefly, urine cfDNA and germline DNA were fragmented to ˜180 bp size fragments prior to library preparation using a LE220-focused ultrasonicator (Covaris, Woburn, Massachusetts). Approximately 32 ng of sheared urine cfDNA or germ line DNA was used for library preparation using the KAPA HyperPrep kit with barcoded adapters containing demultiplexing, deduplicating, and duplexed unique molecular identifiers. Targeted hybrid capture was performed per the standard uCAPPSeq method. A focused MRD gene panel was used spanning 145 kb in size and consisting of 49 consensus driver genes frequently mutated in bladder cancer for the VAF estimation in each sample. For TMB estimation, an expanded panel of 387 kb in size which covers 536 genes was used. Following hybridization capture, libraries were sequenced deeply on a HiSeq 4000 (Illumina, San Diego, California) with 2×150 bp paired-end reads. Sequencing results were analyzed for single nucleotide variants using the CAPP-Seq bioinformatic pipeline. CAPP-Seq was similarly performed on plasma with matched germline DNA.

Single Nucleotide Variant Analysis from cfDNA

Only non-silent mutations with duplex support and with no germline support were considered when querying MRD from cfDNA. Specifically, maximum VAF was defined as the maximum variant allele fraction among all non-silent mutations with duplex support detected by CAPP-Seq using the 145 kb driver gene-focused MRD gene panel, regardless of the number of other mutations detected and their frequencies. Maximum VAF was selected as the metric representing tumor DNA by CAPP-Seq, and was correlated with MRD status in the surgical specimen. Nonsilent SNVs in urine cfDNA with >2.3% VAF3 are represented in the FIG. 4A heatmap. Tumor mutational burden was additionally inferred using the urine CAPP-Seq results. Briefly, the TMB gene panel was utilized, which is 387 kb in size and covers 536 genes, and the equation determined previously by linear regression was applied while accounting for potential dropout in order to infer exome-wide TMB3.

Ultra-Low-Pass Whole Genome Sequencing (ULP-WGS)

ULP-WGS libraries were prepared from 32 to 50 ng of sheared urine cfDNA using the Kapa HyperPrep kit (Roche, Basel, Switzerland). Libraries were balanced, pooled, and sequenced on a HiSeq 4000 (Illumina, San Diego, California) to a median deduplicated depth of 2× (see e.g., TABLE 6). FASTQ files were demultiplexed and raw reads were quality-filtered using fastp v.0.20.0. Quality-filtered reads were then aligned to the hg19 human genome assembly using BWA v.0.7.17. Aligned reads were deduplicated with Samtools v.1.13. ichorCNA v0.2.01515 was then used to infer tumor fractions in each urine cfDNA sample. Briefly, reads were summed in nonoverlapping bins of 106 bases; local read depth was corrected for GC bias and known regions of low mappability, and artifacts were removed by comparison to ichorCNA's built-in healthy control reference. Copy number alterations (CNAs) were then predicted across the whole genome using low tumor fraction parameters for cfDNA samples; X and Y chromosomes were excluded from copy number calculations. ichorCNA then used these binned, bias-corrected copy number values to model a two-component mixture of tumor-derived and non-tumor-derived fragments, from which it inferred the fraction of reads in each sample originating from the tumor (tumor fraction).

The visualization of aggregate genome-wide CNAs (see e.g., FIG. 3A-FIG. 3C) was generated from compiled log₂ ratios of copy number, broken down into three categories: No pathologic complete response (n=39), pathologic complete response (n=35), and healthy adults (n=15). Following the removal of artifacts, regions were classified as exhibiting copy number gain if log₂ of the copy number ratio was >0.58 (log₂ (3/2)) or loss if log₂ of the copy number ratio was <−1.0 (log₂ (½)). Midpoints of genes previously shown to be commonly altered in whole exome sequencing data of muscle-invasive bladder cancer, based on their annotation in the respective TCGA publications are specifically highlighted (see e.g., FIG. 3A-FIG. 3C and FIG. 4A-FIG. 4B).

Machine Learning Model to Predict Pathologic Complete Response and Survival

A random forest model was implemented for the prediction of pCR, which was validated using LOOCV. The maximum VAF, iTMB, and ULP-WGS-inferred tumor fraction (TFx) in urine cfDNA was used, which were combined together into one urine tumor DNA feature for the random forest model via multiplication followed by the square root of the product. Other features in the model included age, gender, ethnicity, smoking status, receipt of neoadjuvant chemotherapy, and tumor invasion status (see e.g., FIG. 8 and FIG. 10). Another LOOCV random forest model was developed using only urine cfDNA features (VAF, iTMB, and TFx) without the clinical variables (see e.g., FIG. 11A-FIG. 11B). The Python scikitlearn package (v0.24.2) was used to implement the random forest algorithm, with the following parameters: n_estimators=2000; criterion=gini; bootstrap=True. The performance of the model after LOOCV for predicting pCR was assessed by receiver operating characteristic (ROC) area under the curve (AUC) analysis. Patients predicted by the LOOCV model to not achieve pCR were defined as MRD-positive, while those predicted to have pCR were defined as MRD-negative. LOOCV model MRD predictions were compared to gold-standard surgical pathology results (see e.g., FIG. 9B) and were also stratified by Kaplan-Meier analysis from the time of surgical resection for progression-free survival (PFS) and overall survival (OS) (see e.g., FIG. 12A-FIG. 12D). The model was additionally generated using independent training and held-out validation cohorts (see e.g., FIG. 15A-FIG. 15B). Furthermore, feature importance levels were calculated by assessing mean decrease in impurity, to determine how classifications of pCR (MRD-negative) versus no pCR (MRD-positive) were affected if a particular feature was left out of the random forest model (see e.g., FIG. 10).

Power and Statistical Analyses

The current study was powered assuming a substantial difference in urine tumor DNA levels between patients who achieved pCR or healthy donors, compared to patients with no pCR. Assuming a large effect size estimated by Cohen's f=0.5, subjects were accrued to this study until there were at least 14 subjects per group (groups=healthy donors, bladder cancer with pCR, bladder cancer with no pCR) in order to detect a difference between healthy or pCR, and no pCR with an estimated power of 80% and significance level of 0.05 as determined by one-way ANOVA. Patient characteristics such as age, gender, ethnicity, smoking history, tumor stage, neoadjuvant chemotherapy, and histology were statistically compared between groups of pCR and no pCR patients using Fisher's exact test for categorical variables and Student's t-test for normally distributed continuous variables (see e.g., TABLE 7). SNV-derived maximum VAFs, inferred tumor mutational burden, and CNA-derived tumor fraction levels in urine cell-free DNA from patients with localized bladder cancer were statistically compared between groups of pCR and no pCR using the Mann-Whitney U-test (see e.g., FIG. 5, FIG. 7A-FIG. 7B, FIG. 6A, FIG. 13A-FIG. 13C, and FIG. 14A-FIG. 14C). The Python scikitlearn package (v0.24.2) was used for random forest modeling with LOOCV (see e.g., FIG. 8 and FIG. 10) or with separate training and validation datasets (see e.g., FIG. 15A and FIG. 15B). ROC analysis was carried out to assess the performance of the LOOCV random forest model and the corresponding AUC was calculated for the full cohort of 74 localized bladder cancer patients with and without pretreatment clinical variables (see e.g., FIG. 9A and FIG. 11B) and for MIBC patients (see e.g., FIG. 13D). MRD predictions based on the LOOCV random forest model were compared to surgical ground-truth by Fisher's exact test (see e.g., FIG. 9B and FIG. 13E). Survival curves for PFS and OS were analyzed by the Kaplan-Meier method and statistical significance was determined by the log-rank test (see e.g., FIG. 12A-FIG. 12D, FIG. 13F-FIG. 13G, FIG. 14D-FIG. 14E, and FIG. 15A-FIG. 15B). The Mantel-Haenszel method was used to estimate hazard ratios. Cox proportional hazards model (PHM) univariate and multivariate analyses were developed to assess both PFS and OS (see e.g., TABLE 8 and TABLE 9). In addition to random forest model prediction, hematocrit, body mass index, and urine cfDNA concentration were included in the multivariate models. For OS, there were no deaths during the follow-up period among patients predicted by the random forest model to achieve pCR. Given this, the assumption of proportional hazards was not met. All Kaplan-Meier and Cox regression analyses were performed starting from the time of surgery. The reverse Kaplan-Meier method was used to calculate the median follow-up time (see e.g., TABLE 1). All statistical analyses were performed using Prism 9 (GraphPad Software, San Diego, California) or SAS version 9.4 (SAS, Cary, North Carolina).

What is claimed is:

1. A method of detecting residual disease in a subject having or suspected of having a urinary tract-associated cancer, the method comprising:
obtaining a urine sample from the subject;
extracting cell-free DNA (cfDNA) from the urine sample;
detecting a cfDNA-derived metric using ultra-low-pass whole genome sequencing (ULP-WGS) and next-generation sequencing (NGS);
wherein the cfDNA-derived metric comprises at least one of a tumor fraction (TFx) value, a variant allele frequency (VAF) value and a tumor mutational burden (TMB) value; and
determining the subject to have residual disease or no residual disease based on the cfDNA-derived metric.

2. The method of claim 1, wherein the NGS comprises urine cancer personalized profiling by deep sequencing (uCAPP-Seq).

3. The method of claim 1, wherein the determining comprises employing a machine learning model based on the cfDNA-derived metric.

4. The method of claim 1, wherein detecting the cfDNA-derived metric further comprises detecting single nucleotide variants (SNVs) or copy number alterations (CNAs) in the cfDNA.

5. The method of claim 1, wherein the cfDNA-derived metric consists of a TFx value, VAF value, and a TMB value.

6. The method of claim 3, wherein the machine learning model comprises a random forest model and is further based on a clinical variable selected from the group consisting of age, gender, ethnicity, smoking status, receipt of chemotherapy, tumor invasion status, and combinations thereof.

7. The method of claim 1, wherein the urinary tract-associated cancer is a bladder cancer.

8. The method of claim 7, wherein the bladder cancer is a muscle-invasive bladder cancer.

9. The method of claim 1, wherein a cancer treatment was administered to the subject prior to obtaining the urine sample, and wherein the cancer treatment is a chemotherapy, a radiotherapy, or an immunotherapy.

10. The method of claim 1, wherein determining the subject to have residual disease comprises determining a negative predictive value (NPV) of at least about 70%, a positive predictive value (PPV) of at least about 60%, or an area under the curve (AUC) of at least about 0.70.

11. The method of claim 3, wherein the machine learning model further predicts overall survival (OS) or progression-free survival of the subject based on the cfDNA-derived metric.

12. A method of treating a subject having or suspected of having a urinary tract-associated cancer, the method comprising:
obtaining a urine sample from the subject;
extracting cell-free DNA (cfDNA) from the urine sample;
detecting a cfDNA-derived metric using ultra-low-pass whole genome sequencing (ULP-WGS) and next-generation sequencing (NGS);
wherein the cfDNA-derived metric comprises at least one of a tumor fraction (TFx) value, a variant allele frequency (VAF) value and a tumor mutational burden (TMB) value; and
determining the subject to have residual disease or no residual disease based on the cfDNA-derived metric; and
providing:
a cancer treatment to the subject if the subject is determined to have residual disease, or
active surveillance to the subject if the subject is determined to have no residual disease.

13. The method of claim 12, wherein the NGS comprises urine cancer personalized profiling by deep sequencing (uCAPP-Seq).

14. The method of claim 12, wherein the determining comprises employing a machine learning model based on the cfDNA-derived metric.

15. The method of claim 12, wherein detecting the cfDNA-derived metric further comprises detecting single nucleotide variants (SNVs) or copy number alterations (CNAs) in the cfDNA.

16. The method of claim 12, wherein the cfDNA-derived metric consists of a TFx value, VAF value, and a TMB value.

17. The method of claim 14, wherein the machine learning model is further based on a clinical variable selected from the group consisting of age, gender, ethnicity, smoking status, receipt of chemotherapy, tumor invasion status, and combinations thereof.

18. The method of claim 12, wherein the urinary tract-associated cancer is a bladder cancer, and wherein the cancer treatment comprises a chemotherapy, a radiotherapy, an immunotherapy, or a surgical treatment.

19. The method of claim 18, wherein the surgical treatment is a cystectomy.

20. The method of claim 12, wherein a cancer treatment was administered to the subject prior to obtaining the urine sample.
